(19) United States
(12) Patent Application Publication
(10) Pub. No.: US 2014/0199650 A1
(43) Pub. Date: Jul. 17, 2014

(54) DENTAL IMPLANTATION SYSTEM AND METHOD USING MAGNETIC SENSORS

(71) Applicant: PRECISION THROUGH IMAGING, INC., Solana Beach, CA (US)

(72) Inventors: Allen M. MOFFSON, Solana Beach, CA (US); Jeffrey A. PRSHA, San Diego, CA (US); Charles E. WHEATLEY, III, Del Mar, CA (US)

(73) Assignee: PRECISION THROUGH IMAGING, INC., Solana Beach, CA (US)

(21) Appl. No.: 14/154,599

(22) Filed: Jan. 14, 2014

Related U.S. Application Data
(63) Continuation of application No. PCT/US2012/046789, filed on Jul. 13, 2012.
(60) Provisional application No. 61/507,956, filed on Jul. 14, 2011.

(51) Int. Cl. A61C 1/08; A61B 5/00; A61B 5/05

(52) U.S. Cl. CPC A61C 1/082; A61B 5/05; A61B 5/682
USPC 433/27; 600/409

(57) ABSTRACT

Provided herein, inter alia, is a system for indicating the location of a dental drill includes a dental handpiece, which further includes the dental drill. A plurality of sensors detect a magnetic field and produce a set of outputs, which are usable at least in part to indicate the location of the dental drill. The sensor outputs may be processed to produce an indication of the spatial relationship of the drill to a patient's dentition. The indication is preferably graphical, and may be presented to a dental professional using the system during an implant procedure to provide visual feedback about the procedure. The indication may be repeatedly updated, substantially in real time.

This application is a continuation of PCT International Patent Application No. PCT/US2012/046789, filed Jul. 13, 2012, which in turn claims priority from U.S. Provisional Patent Application No. 61/507,956 filed Jul. 14, 2011 and titled “Dental Implantation System and Method Using Magnetic Sensors”, the entire disclosures of each of which are hereby incorporated by reference herein for all purposes.

### BACKGROUND

Dental implant surgery involves placing a prosthetic device such as one or more artificial replacement teeth in the mouth of a patient. Such prosthetic devices must be precisely placed in the mouth for the best aesthetic and functional results. Precise placement of the prosthetic device requires suitable preparation of the implant site with respect to surrounding tissue and bone. The prosthetic device typically comprises a tooth implant abutment, a pontic attached thereto, and a tooth implant fixture that extends from the abutment and is received into an implant shaft drilled into the patient's bone with a drilling tool (e.g., dental handpiece). During the drilling of bone to create the implant shaft, great care must be taken to avoid causing injury to the patient. Injury may be caused by, for example, inadvertent entry into the mandibular nerve canal, inadvertent entry into the sinuses, perforation of the cortical plates, damage to adjacent teeth, or other damage known in the art.

Systems that provide real-time imaging of implant sites can be helpful to the implant practitioner in avoiding injury to patients and in more accurately preparing the bone and implant site, and preparing of the shaft for receiving the implant. Conventional systems that provide such imaging can be cumbersome, complicated, and difficult to use. Moreover, the images provided by systems that rely on optical (viewable) images can be limited by images that are obscured by fluids, including blood and water found at the implant site during drilling. In addition, some computer-assisted imaging systems are not especially accurate in determining location of anatomical structures and instruments, nor are they especially accurate in updating such location information in real-time during the drilling procedure.

Improved real-time imaging would assist the implant practitioner with precise location of the drilling tool during the procedure and would benefit the patient by reducing the risk of injury and helping to provide an effective implant. Such techniques could also be used in a variety of procedures, beyond the dental field, including, for example, other health practices and non-medical procedures.

### BRIEF SUMMARY

According to one aspect, a system for indicating the location of a dental drill comprises a dental handpiece including the dental drill, and a plurality of sensors that detect a magnetic field. The sensors produce a set of respective sensor outputs, and the sensor outputs are usable at least in part to indicate the location of the dental drill.

According to another aspect, a method of indicating the location of a dental drill comprises reading outputs produced by a set of sensors, wherein the sensors detect a magnetic field, and wherein the sensor outputs are usable to detect the location of a dental drill in relation to the sensors. The method further comprises processing the sensor outputs to produce an indication of the spatial relationship of the dental drill to a patient's dentition, and displaying the indication of the spatial relationship of the dental drill to the patient's dentition.

According to another aspect, a workpiece guide comprises a dental arch portion that conforms to the dentition of a particular patient, and a set of sensors fixed in relation to the dental arch portion. Each sensor is capable of producing an output that indicates at least one characteristic of a magnetic field.

According to another aspect, a method comprises fabricating a workpiece guide of a configuration to engage the dentition of a particular patient having an implant site, and placing a set of fiducial references on the workpiece guide. The method further comprises fixing a sensor to the workpiece guide. The sensor is capable of, when the sensor is exposed to a magnetic field, producing an output indicating an aspect of the magnetic field.

According to another aspect, a computerized controller comprises an image processor that receives a radiographic image of a patient's dentition, and a location system that receives outputs from one or more sensors. The sensors detect at least one aspect of a magnetic field, and the sensor outputs change as the spatial relationship of the magnetic field and the sensors changes due to changes in the location of a dental handpiece that includes a dental drill. The location system processes the sensor outputs to determine the location of the dental drill in relation to the patient's dentition. The computerized controller further includes a viewing system that generates a display image at a computer display such that the generated display image comprises the image of the patient's dentition and a depiction of the location of the dental drill relative to the patient's dentition as determined by the location system.

According to another aspect a computerized controller comprises a processor, a data input interface, a display, and a computer-readable memory. The computer readable memory holds instructions that, when executed by the processor, cause the computerized controller to read outputs produced by a set of sensors. The sensors detect a magnetic field and the sensor outputs are usable to characterize the spatial relationship of a dental drill to the sensors. The instructions, when executed by the processor, further cause the computerized controller to process the outputs to produce an indication of the spatial relationship of the dental drill to a patient's dentition, and display the indication of the spatial relationship of the dental drill to the patient's dentition.

According to another aspect, a calibration station comprises a body defining a receptacle. The receptacle is of a shape and size to receive a dental drill. The calibration station further includes a plurality of sensors surrounding the receptacle, each sensor capable of producing an output when the sensor is exposed to a magnetic field associated with a dental drill placed in the receptacle.

According to another aspect, a non-transitory computer readable medium holds computer instructions adapted to be executed to implement a method of indicating the location of a dental drill. The method includes reading outputs produced by a set of sensors. The sensors detect a magnetic field, and the sensor outputs are usable to detect the location of a dental drill in relation to the sensors. The method also includes processing the sensor outputs to produce an indication of the spatial relationship of the dental drill to a patient's dentition, and displaying the indication of the spatial relationship of the dental drill to the patient's dentition.

According to another aspect, a sensing device includes a carrier having circuit traces, the carrier defining a through hole. The sensing device also includes a plurality of electronic sensors mounted to the carrier around the through hole. Each sensor is sensitive to a magnetic field and configured to produce an output indicating an aspect of the magnetic field. The sensing device is of a size and shape for the sensors to fit within the mouth of a dental patient.

According to another aspect, a kit includes a sensing device. The sensing device includes a carrier having circuit traces, the carrier defining a through hole, and a set of electronic sensors mounted to the carrier around the through hole. Each sensor is sensitive to a magnetic field and configured to produce an output indicating an aspect of the magnetic field. The sensing device is of a size and shape for the sensors to fit within the mouth of a dental patient. The kit further includes a non-transitory computer readable medium holding computer instructions adapted to be executed to implement a method of indicating the location of a dental drill. The method includes reading outputs produced by the set of sensors, wherein the sensors detect a magnetic field, and wherein the sensor outputs are usable to detect the location of a dental drill in relation to the sensors. The method further includes processing the sensor outputs to produce an indication of the spatial relationship of the dental drill to a patient's dentition, and displaying the indication of the spatial relationship of the dental drill to the patient's dentition.

### BRIEF DESCRIPTION OF THE DRAWINGS

FIG. 1 illustrates a system in accordance with an embodiment of the invention, for indicating the location of a dental drill.

FIG. 2 illustrates a block diagram of an exemplary controller.

FIG. 3 is a block diagram illustrating the interaction of components of a system, in accordance with embodiments.

FIG. 4 illustrates a step in the fabrication of a workpiece guide, in accordance with embodiments.

FIG. 5 illustrates one simplified example interactive user interface by which a dental professional may determine and specify a desired implant shaft.

FIG. 6 illustrates the example workpiece guide of FIG. 4, in a later stage of fabrication.

FIG. 7 illustrates an example calibration station, according to embodiments of the invention.

FIG. 8 is a block diagram of a system in accordance with other embodiments.

FIG. 9 illustrates an example arrangement of components that may reside in a patient's mouth during an implant procedure.

FIGS. 10A-10C illustrate an example magnetizer/calibration station, in accordance with embodiments.

FIG. 11 illustrates one example technique for determining the location of a drill with respect to sensors, and thus with respect to the patient's dentition.

FIG. 12 illustrates a system in accordance with another embodiment of the invention, for indicating the location of a dental drill.

FIG. 13A illustrates a workpiece guide and a sensor assembly, in accordance with embodiments of the invention.

FIG. 13B shows the sensor assembly of FIG. 13A in more detail.

FIG. 13C shows the sensor assembly of FIG. 13A engaged with alignment pins on a workpiece guide, in accordance with embodiments of the invention.

FIG. 13D shows the sensor assembly of FIG. 13A engaged with different alignment pins.

FIG. 14A illustrates a workpiece guide and a sensor assembly, in accordance with other embodiments of the invention.

FIG. 14B shows the sensor assembly of FIG. 14A in place over the workpiece guide.

FIG. 15 shows the relationship of a magnetic field with sensors in a “dual quad” arrangement, in accordance with embodiments of the invention.

FIG. 16 illustrates a coordinate system useful in describing sensor behavior.

FIG. 17 illustrates an orthogonal view of the interaction of the field and sensors of FIG. 15, in more detail.

FIG. 18 shows an approximate representation of a field angle.

FIG. 19 is a flowchart of a method according to an example embodiment.

### DETAILED DESCRIPTION

Unless expressly defined, the terms used herein have meanings as customarily used in the dental and medical arts.

The terms “implant,” “dental implant” and the like (noun), refer in the customary sense to a permanently placed (e.g., non-removable or difficult to remove) prosthetic device which includes an artificial tooth root replacement. In some embodiments, the implant includes an implant fixture which is embedded in bone and undergoes integration (i.e., osseointegration) to form a stable integrated structure capable of supporting an artificial tooth or providing support for another dental structure including, for example but not limited to, an implant-support bridge or implant-supported denture, as known in the art. The implant fixture is joined to an implant abutment, typically near the gingival surface, to which implant abutment can be affixed a replacement tooth (i.e., pontic). The term “implant” (verb) refers in the customary sense to the placement of a dental implant. “Implant fixture” refers to that portion of a dental implant which is embedded in bone or other hard tissue or material and which serves to anchor the implant, as known in the art.

The term “patient” refers to a recipient of dental attention, care, or treatment. In some embodiments, a patient is a mammal, for example a human, but a patient may also be an animal other than a human.

The term “dentition” refers to the arrangement of teeth in the mouth. An image of a patient's dentition may show all or part of the patient's dentition, and need not depict all of the patient's teeth.

“Workpiece guide” refers in the customary sense to a removable prosthetic guide capable of being rigidly affixed within the mouth of a patient to the upper or lower dental arch. A workpiece guide may have one or more radiopaque markers affixed thereto. A workpiece guide may be formed on an impression of the patient's dentition and/or other structural features of the mouth by methods well known in the art. A workpiece guide may be fabricated from a variety of materials, including but not limited to, thermosetting and light-setting plastics, acrylic, and the like, as known in the art.

The terms “radiopaque marker,” “radiopaque fiducial marker,” “fiducial marker” and the like refer in the customary sense to a deposit of radiopaque material on and/or within, for example, a radiographic guide, capable of being located in a radiographic image. A “fiducial reference” is a reference locator for a part, and may be, for example, a radiopaque fiducial marker or a mechanical datum.

“Implant site” refers to an oral site capable of receiving, or having received, an implant.

“Implant drill shaft,” “implant shaft” and the like in the context of dental implantation refer to a hole which is formed to receive an implant fixture. Such a hole may also be referred to as an “osteotomy site” in the art. “Desired implant shaft,” “proposed implant shaft” and the like refer to the location (i.e., position, depth and angular orientation relative to anatomical structures of the patient identified e.g., in a 3-D scan image) of an implant shaft to be drilled.

“Handpiece” and “dental handpiece” refer in the customary sense to a dental drilling device suitable for drilling dental tissue. In some embodiments, a dental handpiece may include a handle, a handpiece head, a drill engine contained therein, and a drill attached to the drill engine.

“Drill” refers in the customary sense to a dental drill having a drill shaft, optionally a drill shaft extension, and a drill tip. Types of drill tip include burr, conical, twist and the like, as known in the art. In one embodiment, a drill shaft extension is non-magnetic. In one embodiment, a drill shaft extension is magnetic, preferably having the same magnetic properties as the drill tip to which it is attached.

Additional information may be found in co-pending International Patent Application PCT/US11/22290, filed Jan. 24, 2011 and titled “Dental Implantation System and Method”, the entire disclosure of which is hereby incorporated by reference herein for all purposes.

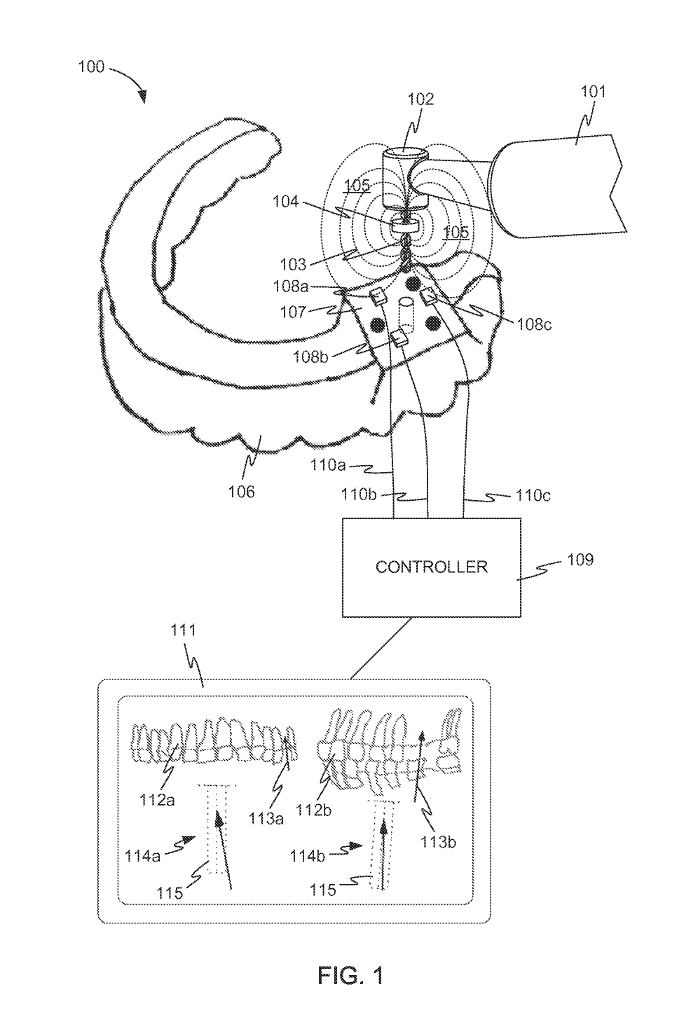

FIG. 1 illustrates a system 100 in accordance with an embodiment of the invention, for indicating the location of a dental drill. For the purposes of this disclosure, the term “location” encompasses angular orientation as well as translational position.

In example system 100, a dental handpiece 101 includes a handpiece head 102, which may house a motor or other drill engine, which in turn drives drill 103 mounted to dental handpiece 101. A magnetized element 104 is fixed to drill 103, and generates a magnetic field 105. In the example shown, magnetized element 104 is toroidal in shape and generates a lobed magnetic field, but it is contemplated that other kinds of magnetized elements and field shapes may be used. For example, in some embodiments, the drill 103 itself may be magnetized and serve as the magnetized element. In other embodiments, magnetic field 105 may be transverse to drill 103, and in some embodiments may have multiple poles. Many field shapes are possible. In any event, the generated magnetic field and drill 103 should remain in a fixed spatial relationship with respect to each other, so that as dental handpiece 101 and drill 103 are moved, the magnetic field moves with them.

A workpiece guide 106 is also provided. Workpiece guide 106 is molded to conform to the dentition of a particular implant patient, and may be made of any suitable material such as a thermosetting or light setting polymer. Workpiece guide 106 preferably conforms to at least part of an upper or lower dental arch of the patient, and may encompass an implant site where an implant is to be placed. In some embodiments, workpiece guide 106 conforms to the entire dental arch, and in other embodiment, workpiece guide 106 conforms to only part of the dental arch. Workpiece guide 106 preferably is removable from and replaceable onto the patient's dentition, but conforms tightly to the patient's teeth so that when replaced, it returns repeatably enough to the same location that any errors introduced by the removal and replacement are negligible. Workpiece guide 106 may conveniently include a relatively flat surface 107 over the implant site, but this is not a requirement.

Affixed to workpiece guide 106 are sensors 108a, 108b, and 108c. While a constellation of three sensors 108a-108c is shown, workable systems may be envisioned having more sensors (e.g. 4, 5, 6, 7, 8, or even more sensors) or fewer sensors (e.g. 2 sensors). For the purposes of this disclosure, a “constellation” of elements is a set of elements in an arrangement fixed in relation to each other. Each of sensors 108a-108c detects at least one aspect of magnetic field 105, and produces an output (also referred to as a sensor output) that changes as the spatial relationship between the sensor and magnetized element 104 changes due to changes in the location of dental handpiece 101 and consequent changes in the location of magnetic field 105. Each of sensors 108a-108c may be, for example, a model HMC5883L 3-Axis Digital Compass integrated circuit available from Honeywell International Inc., of Morristown, N.J., USA. When exposed to a magnetic field, such a sensor provides output that describes the strength of the local magnetic field, and the direction of the magnetic field in relation to the axes of the sensor.

In other embodiments, the positions of the sensors and magnetized element may be reversed. For example, a magnetized element may be fixed to workpiece guide 106, and a set of sensors fixed to handpiece 101.

The shape of magnetic field 105 is known, and the spatial relationship of magnetic field 105 to drill 103 may be characterized ahead of time. A sufficient number of sensors, which may be one or more sensors, is provided that the location of magnetic field 105 with respect to the sensors can be determined given the sensor outputs and knowledge of the shape of magnetic field 105. That is, the sensor outputs characterize the location of the magnetic field in relation to the sensors. The “location” of the magnetic field may be conceptualized as the collective locations in space of the field lines of the magnetic field. In some embodiments, redundant sensors may be provided. For example, if two sensors are sufficient to characterize the location of magnetic field 105, three sensors may be provided so that if the location determined from the outputs of any pair sensors differs from the location determined from the outputs of any other pair, it may be assumed that an error has occurred and the user of the system may be alerted to avoid possible injury to the patient.

Once the location of magnetic field 105 is determined from the sensor outputs, the location of drill 103 in relation to sensors 108a-108c can be determined from the previously-characterized spatial relationship of magnetic field 105 to drill 103.

Preferably, the spatial relationship of sensors 108a-108c to the patient's dentition has also been previously characterized (as is explained in more detail below), and therefore the location of drill 103 with respect to the patient's dentition can be computed. In example system 100, a computerized controller 109 receives the sensor outputs 110a-110c. Controller 109 also stores information describing the previously-determined shape of magnetic field 105, and the previously-characterized spatial relationships between magnetic field 105 and drill 103, and between sensors 108a-108c and the patient's dentition. Controller 109 may further store a previously-recorded image or model of the patient's dentition, for example an x-ray image or a three-dimensional model constructed from data gathered by computerized axial tomography, also known as a CAT scan or CT scan.

During use of system 100, controller 109 may repeatedly read sensor outputs 110a-110c and compute the spatial relationship between drill 103 and the patient's dentition. The relationship is preferably presented to the user in a graphical representation on a visual display 111. Display 111 may be, for example, a cathode ray tube, a liquid crystal display, or another kind of device capable of providing a graphical display.

In the example shown, display 111 shows previously-recorded images 112a and 112b, which are pictorial representations of the patient's dentition. For example, images 112a and 112b may be digitized x-ray images or may be derived from CT scan images. Superimposed on images 112a and 112b are arrows 113a and 113b, which represent the current location of drill 103 in relation to the patient's dentition. While images 112a and 112b may be static, arrows 113a and 113b are dynamically updated, preferably substantially in real time, to give the dental professional using the system visual feedback of the location of drill 103 with respect to the patient's dentition. Such visual feedback may assist the dental professional in avoiding errors or injury to the patient. For the purposes of this disclosure “substantially in real time” means that updates are performed often enough and with little enough delay that the display reflects movements of handpiece 101 with little or negligible delay, and the dental professional's control of handpiece 101 is not significantly compromised by measurement or processing delays. In some embodiments, the measurement and processing delays may be imperceptible. Because the sensing used to determine the drill position is done magnetically, it is typically insensitive to liquids or biological particulates that may be present at the implant site and that might obscure direct viewing of the implant site.

While example images 112a and 112b show front and side views of the patient's dentition, other appropriate views may be utilized. In some embodiments, a three-dimensional model of the patient's dentition may be used, and the user of the system may be able to rotate or otherwise reorient the displayed model to obtain a more convenient view. Any representations of the drill location such as arrows 113a and 113b would be simultaneously redrawn so as to show their correct locations in the displayed model.

Also shown in the example display 111 are indications 114a and 114b of the spatial relationship of drill 103 with a previously-specified desired implant shaft 115. Determination of the desired implant shaft is described in more detail below. Controller 109 utilizes the specification of desired implant shaft 115 and the computed location of drill 103 to generate indications 114a and 114b. Controller 109 may also alert the user if the location of drill 103 departs from desired implant shaft 115 more than a predetermined amount. For example, controller 109 may alert the user if the location of the tip of drill 103 departs from the centerline of desired implant shaft by more than 0.1 millimeters, 0.3 millimeters, 0.5 millimeters, 1.0 millimeter, or another predetermined amount. In some embodiments, controller 109 may alert the user if the angular orientation of drill 103 departs from the centerline of desired implant shaft 115 by more than 0.2 degrees, 0.5 degrees, 1 degrees, 2 degrees, 3 degrees, or by another predetermined amount. Many other techniques for measuring departure of the location of drill 103 from desired implant shaft 115 are possible.

To alert the user of a departure from desired implant shaft 115, controller 109 may generate a warning signal such as visual signal, an audio signal, both a visual signal and an audio signal, or a signal of another kind. For example, an alarm may sound to warn the user of a departure, and in some embodiments, the pitch or volume of the alarm may be varied to indicate the severity of the departure. In other embodiments, some part of display 111 may be altered to visually indicate a departure. For example, desired implant shaft 115 could be depicted in red when a departure occurs, and could be depicted in green when drill 103 is properly located with respect to desired implant shaft 115. Many other kinds of warning signals are possible.

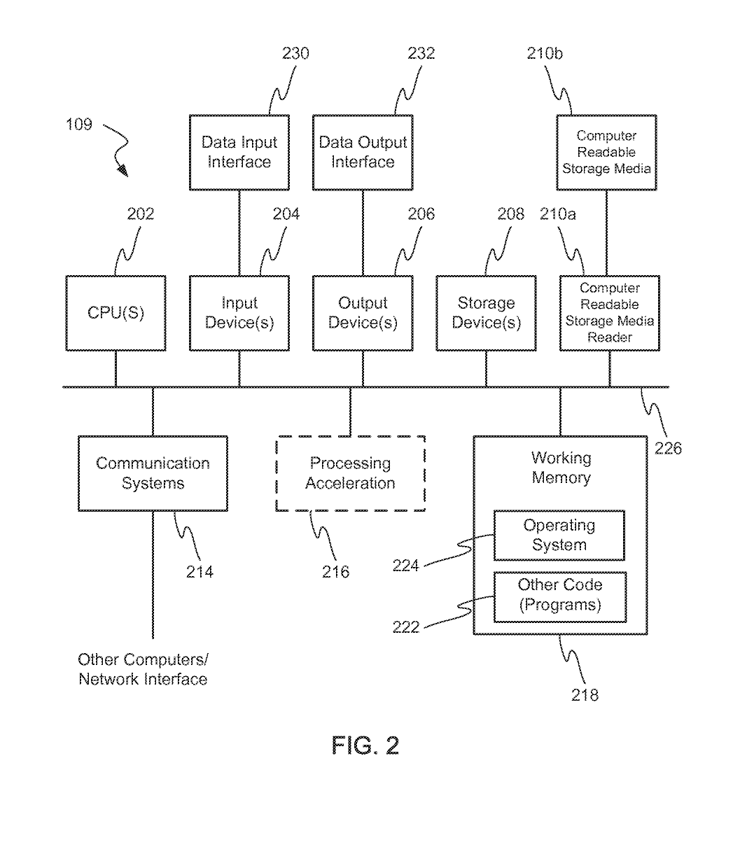

FIG. 2 illustrates a block diagram of an exemplary controller 109. It should be noted that FIG. 2 is meant only to provide a generalized illustration of various components, any or all of which may be utilized as appropriate. FIG. 2, therefore, broadly illustrates how individual system elements may be implemented in a relatively separated or relatively more integrated manner.

Controller 109 is shown comprising hardware elements that can be electrically coupled via a bus 226 (or may otherwise be in communication, as appropriate). The hardware elements can include one or more central processor units (CPUs) 202, including without limitation one or more general-purpose processors and/or one or more special-purpose processors or processor cores. The hardware elements can further include one or more input devices 204, such as a computer mouse, a keyboard, a touchpad, and/or the like for providing user input to the CPU 202; and one or more output devices 206, such as a flat panel display device, a printer, visual projection unit, and/or the like. Data input interface 230 preferably also includes an interface for receiving sensor outputs 110a-110c from sensors 108a-108c. For example, sensor outputs 110a-110c may be analog signals that are converted to digital signals by controller 109, or may be digital signals communicating numerical values. Sensor outputs 110a-110c may be received over a wire or cable in some embodiments. In other embodiments, sensor outputs 110a-110c may be received over a wireless link, for example via a Bluetooth interface, a Zigbee interface, or other kind of standard or proprietary wireless interface.

Controller 109 may further include (and/or be in communication with) one or more storage devices 208, which can comprise, without limitation, local and/or network accessible storage and/or can include, without limitation, a disk drive, a drive array, an optical storage device, solid-state storage device such as a random access memory (“RAM”), and/or a read-only memory (“ROM”), which can be programmable, flash-updateable, and/or the like.

Controller 109 can also include a communications subsystem 214, which can include without limitation a modem, a network card (wireless or wired), an infra-red communication device, a wireless communication device and/or chipset (such as a Bluetooth device, an 802.11 device, a WiFi device, a WiMax device, cellular communication facilities, etc.), and/or the like. The communications subsystem 214 may permit data to be exchanged with other computers, with a network via a network interface, and/or any other external devices described herein. In many embodiments, controller 109 will further include a working memory 218, which can include RAM and/or ROM devices, as described above.

Controller 109 also may include software elements, shown as being located within the working memory 218. The software elements can include an operating system 224 and/or other code, such as one or more application programs 222, which may comprise computer programs that are supported by the operating system for execution, and/or may be designed to implement methods described herein and/or configure systems as described herein. Merely by way of example, one or more procedures described with respect to the method(s) discussed above might be implemented as code and/or instructions executable by a computer (and/or a processor within a computer) such as controller 109. A set of these instructions and/or code might be stored on a computer readable storage medium 210b. In some embodiments, the computer readable storage medium 210b is the storage device(s) 208 described above. In other embodiments, the computer readable storage medium 210b might be incorporated within a computer system. In still other embodiments, the computer readable storage medium 210b might be separate from the computer system (i.e., it could be a removable medium, such as a compact disc, optical disc, flash memory, etc.), and or provided in an installation package, such that the storage medium can be used to program a general purpose computer with the instructions/code stored thereon. These instructions might take the form of executable code, which is executable by controller 109 and/or might take the form of source and/or installable code, which, upon compilation and/or installation on controller 109 (e.g., using any of a variety of generally available compilers, installation programs, compression/decompression utilities, etc.), then takes the form of executable code. In these embodiments, the computer readable storage medium 210b may be read by a computer readable storage media reader 210a of controller 109.

The various components of controller 109 communicate with each other via a system bus 226. Optional processing acceleration 216 may be included in the computer system, such as digital signal processing chips or cards, graphics acceleration chips or cards, and/or the like. Such processing acceleration may assist the CPU 202 in performing the functions described herein with respect to providing the display images.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

In some embodiments, one or more of the input devices 204 may be coupled with a data input interface 230. For example, the data input interface 230 may be configured to directly interface with sensors 108a-108c, whether physically, optically, electromagnetically, or the like. Further, in some embodiments, one or more of the output devices 206 may be coupled with data output interface 232. The data output interface 232 may be configured, for example, to produce data suitable for controlling tools or processes associated with the implant procedure, such as CAD/CAM systems or device manipulation and control systems.

In one embodiment, some or all of the display functions described herein are performed by controller 109 in response to the CPU 202 executing one or more sequences of one or more instructions (which might be incorporated into the operating system 224 and/or other code, such as an application program 222) contained in the working memory 218. Such instructions may be read into the working memory 218 from another machine-readable medium, such as one or more of the storage device(s) 208 (or 210). Merely by way of example, execution of the sequences of instructions contained in the working memory 218 might cause the processor(s) 202 to perform one or more procedures of the methods described herein.

The terms “machine readable medium” and “computer readable medium,” as used herein, refer to any medium that participates in providing data that causes a machine to operate in a specific fashion. In an embodiment implemented using controller 109, various machine-readable media might be involved in providing instructions/code to processor(s) 202 for execution and/or might be used to store and/or carry such instructions/code (e.g., as signals). In many implementations, a computer readable medium is a physical and/or tangible storage medium. Such a medium may take many forms, including but not limited to, non-volatile media, volatile media, and transmission media. Non-volatile media includes, for example, optical or magnetic disks, such as the storage device(s) (208 or 210). Volatile media includes, without limitation, dynamic memory, such as the working memory 218. Transmission media includes coaxial cables, copper wire, and fiber optics, including the wires that comprise the bus 226, as well as the various components of the communication subsystem 214 (and/or the media by which the communications subsystem 214 provides communication with other devices).

Common forms of physical and/or tangible computer readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, an EPROM, a FLASH-EPROM, any other memory chip or cartridge, a carrier wave as described hereinafter, or any other medium from which a computer can read instructions and/or code. A “non-transitory computer readable medium” is a medium in which data can reside more than fleetingly. A non-transitory computer readable medium may require that power be supplied to it. Examples of non-transitory computer readable media include, without limitation, ROM, RAM, machine registers, EPROM, FLASH-EPROM, various kinds of disk and tape storage, and the like.

Various forms of machine-readable media may be involved in carrying one or more sequences of one or more instructions to the CPU 202 for execution. Merely by way of example, the instructions may initially be carried on a magnetic disk and/or optical disc of a remote computer. A remote computer might load the instructions into its dynamic memory and send the instructions as signals over a transmission medium to be received and/or executed by controller 109. These signals, which might be in the form of electromagnetic signals, acoustic signals, optical signals, and/or the like, are all examples of carrier waves on which instructions can be encoded, in accordance with various embodiments of the invention.

The communications subsystem 214 (and/or components thereof) generally will receive the signals, and the bus 226 then might carry the signals (and/or the data, instructions, etc. carried by the signals) to the working memory 218, from which the processor(s) 202 retrieves and executes the instructions. The instructions received by the working memory 218 may optionally be stored on a storage device 208 either before or after execution by the CPU 202.

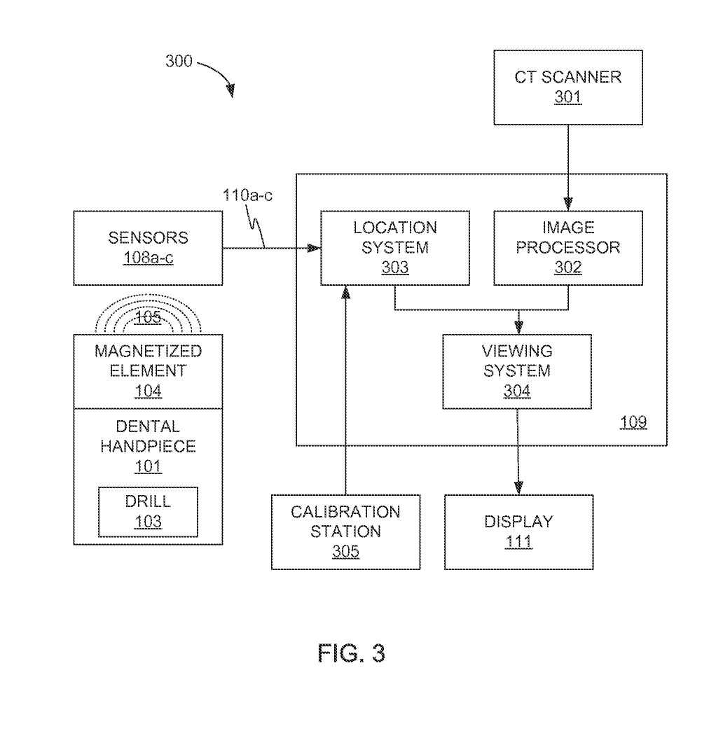

FIG. 3 is a block diagram illustrating the interaction of components of a system 300, in accordance with embodiments. A CT scanner 301 may be used to capture a radiologic image of the patient's dentition and a workpiece guide such as workpiece guide 106. The image may be passed to an image processor 302 for storage and analysis. Magnetized element 104 attached to dental handpiece 101 generates magnetic field 105, which is sensed by sensors 108a-108c. Sensors 108a-108c produce outputs 110a-110c, which pass to a location system 303 of controller 109. Location system 303 may also receive information from image processor 302, and computes an indication of the spatial relationship of drill 103 to the patient's dentition. Information from image processor 302 and location system 303 is passed to viewing system 304, which may construct a composite image showing the patient's dentition and a representation of the location of drill 103. The composite image may then be displayed on display 111. The system may further include a calibration station 305 usable to characterize the spatial relationship of magnetic field 105 and drill 103, as will be discussed in more detail below.

The sequence of events leading to the placement of a dental implant follows a path determined by the professional judgment and practice of the implant practitioner. A typical sequence is described below.

Presentation.

A patient in need of an implant would present for evaluation to a dental practitioner trained in the art of implantology (i.e., “an implant practitioner”). The terms “implantology” and the like refer in the customary sense to the practice of dentistry related to placing dental implants. Typically, the patient will have been referred by a general dentist, prosthodontist, restorative dentist, periodontist, or other practitioner as the result of a perceived need for an implant. A variety of needs for an implant are recognized in the art, including but not limited to replacing one or more teeth, providing an abutment to anchor a dental prosthesis, and in the extreme case of an edentulous patient, actually providing the sole anchoring means for a denture, bridge, or other dental prosthesis.

Evaluation.

Patient evaluation determines whether a patient is a candidate for an implant. Evaluation considerations, in the professional judgment of the dental practitioner, include a variety of factors, including but not limited to, the general and oral health of the patient, medications currently taken by the patient, the site of the implant, proximity to adjacent teeth, and the positioning and morphology of adjacent anatomical landmarks including, but not limited to, the sinus and nasal passages and the floors thereof, other bony and nervous system features of the mandible or maxilla, the mental foramen, adjacent teeth, and available bone. The term “available bone” as used herein refers to tissue into which an implant may be placed. Available bone may include only naturally occurring bone, or may include additional material placed by a dentist to enhance the stability of an implant. A variety of methods for enhancing available bone are known in the art, including but not limited to, sinus lifting and bone grafting. Very high accuracy is required in dental implantology, where even a fraction of a millimeter of excess penetration, for example of the maxillary or mandibular tissue, or a small angular misalignment can mean the difference between a successful and an unsuccessful procedure.

Patient evaluation can include acquiring and analyzing one or more conventional X-ray images (i.e., “screening X-rays”), as known in the art. Due to the limitations of 2-dimensional screening X-rays, the amount of available bone may not be known to the implant practitioner upon viewing only the screening X-rays. Those skilled in the art will know that multiple X-ray scans comprising a 3-dimensional radiographic scan, such as a CT scan, can provide a 3-dimensional view of anatomical structures. Accordingly, a 3-dimensional radiographic scan of the patient is desirable for at least the purpose of evaluation with respect to, for example, the amount of available bone.

Fabrication of Workpiece Guide.

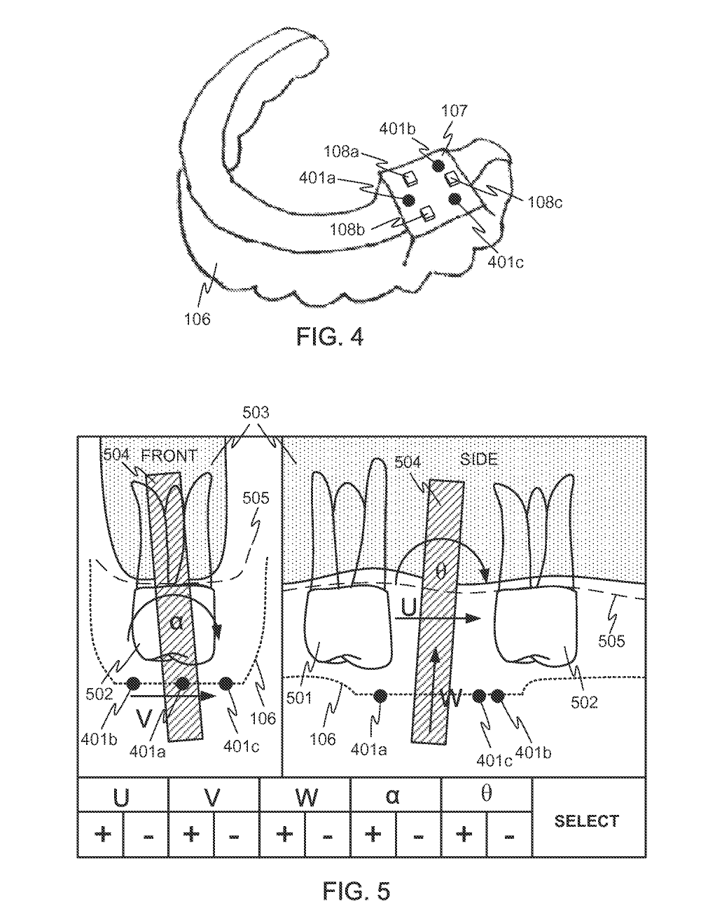

In an initial step, workpiece guide 106 is fabricated as shown in FIG. 4. The fabrication of workpiece guide 106 may be done according to known methods. For example, a cast of the patient's dentition may be made and the guide molded to the cast. Workpiece guide 106 conforms to an upper or lower dental arch of the patient, and may encompass the implant site. Additional methods for the fabrication of a workpiece guide are known in the art including, but not limited to, computer assisted manufacturing processes based on a previously obtained 3-dimension radiographic scan. The initial radiographic workpiece guide is preferably sufficiently sturdy to resist flexing under operation of the handpiece during dental surgery including implant placement.

At least three radiopaque fiducial markers, e.g., 401a, 401b, and 401c, may be fixed to workpiece guide 106. In the example of FIG. 4, fiducial markers 401a-401c are shown as fixed or embedded in relatively flat surface 107 over the implant site, but this is not a requirement. The fiducial markers, e.g. 401a-401c, must be non-collinear in order to define a plane in 3-dimensional space, but otherwise can be placed in any convenient locations on workpiece guide 106. In other embodiments, fiducial references other than radiopaque fiducial markers may be used. For example, workpiece guide 106 may include a set of mechanical datums sufficient to define the locations of features of workpiece guide 106.

Sensors 108a-108c may be placed on workpiece guide 106 at this stage, or may be placed at a later time. Preferably, sensors 108a-108c are positioned to receive adequate signals from a magnetic field such as magnetic field 105 during drilling. While sensors 108a-108c are shown without interconnecting wires for clarity of illustration, in actual embodiments, sensors 108a-108c may be fixed to a printed circuit board or flex circuit that is in turn fixed to workpiece guide 106, such as by an adhesive, to hold sensors 108a-108c in fixed relationship to workpiece guide 106. Sensors 108a-108c are preferably positioned in a known relationship to the fiducial references of workpiece guide 106, for example radiopaque fiducial markers 401a-401c, and that relationship is characterized for future reference. In some embodiments, sensors 108a-108c may serve as the radiopaque fiducial markers.

Three-Dimensional Imaging.

Workpiece guide 106 is then engaged with the patient's dentition, and a radiographic image of the workpiece guide and the patient's dentition is obtained while the patient is wearing the workpiece guide. For example, the radiographic image may be obtained by a CT scan, and preferably shows details of the patient's dentition, as well as of workpiece guide 106. Fiducial markers 401a-401c are radiopaque, and will show clearly in the radiographic image. Because of the repeatable fit of workpiece guide 106 with the patient's dentition and the fact that fiducial markers 401a-401c are fixed to workpiece guide 106, fiducial markers 401a-401c (or other fiducial references) may serve as an anchor reference in relation to the patient's dentition.

Determining Implant Location.

A dental professional, for example the implant practitioner, then determines the desired location of the implant shaft. This may be done, for example, by examining a three-dimensional model of the patient's dentition and bone structure derived from the CT scan. The dental professional specifies the location of the desired implant shaft, including its position, angular orientation, and depth, in relation to the patient's dentition, and therefore in relation to fiducial markers 401a-401c or other fiducial references.

FIG. 5 illustrates one simplified example interactive user interface by which a dental professional may determine and specify the desired implant shaft. In the example of FIG. 5, a computer system, possibly controller 109 or another computer system has constructed a three-dimensional model from CT scan data, and displayed portions of the model, including teeth 501 and 502, bone 503, and workpiece guide 106. Radiopaque fiducial markers 401a-401c are also visible. The model and display may be similar to those commonly used in computer aided design (CAD) systems that perform three-dimensional modeling. The system also superimposes a representation of an implant shaft 504. The different structures such as bone 503, teeth 501 and 502, a visible gumline 505, and workpiece guide 106, may be distinguished in the display by different colors, textures, degrees of opacity, or other means, for example according to their relative density or opacity to x-ray radiation. The dental professional can then manipulate the implant shaft representation 504 using keyboard or mouse clicks to translate and rotate the implant shaft representation 504 and adjust its depth, until a location is reached that, in the judgment of the dental professional, will most likely result in a successful implant.

In some embodiments, additional views or controls may be provided for viewing and magnifying different portions of the patient's dentition, for changing the angle of view displayed, or for other functions that may assist the dental professional in locating a desired implant shaft location. Views need not be displayed orthogonally. Many other suitable user interfaces may be envisioned.

Once the dental professional is satisfied, he or she may “select” the location, or otherwise indicate that the displayed implant shaft representation 504 is in the desired position. The computer system may then record the mathematical description of the shaft location. The locations of radiopaque fiducial markers 401a-401c are also determined from the three-dimensional model, and thus the spatial relationship of the desired implant shaft and the radiopaque fiducial markers 401a-401c can be mathematically characterized.

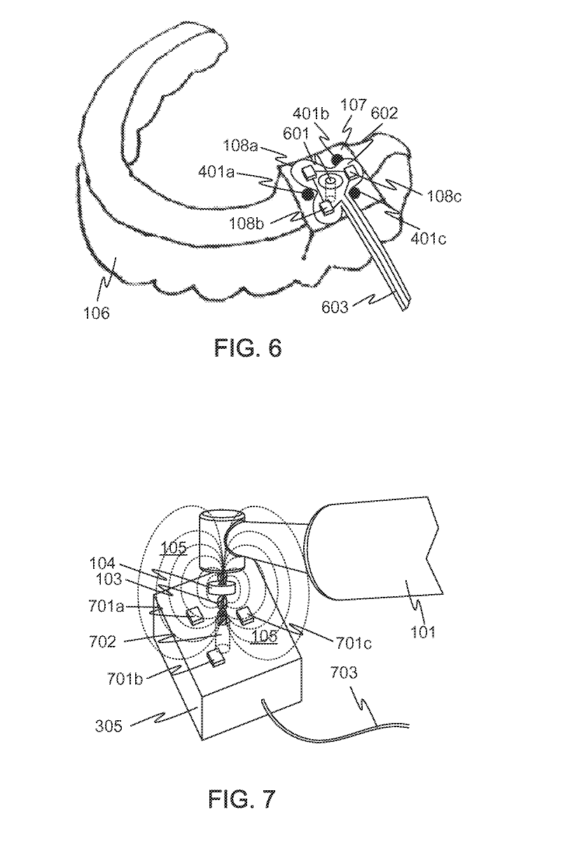

In some embodiments, a pilot hole 601 may then be formed in workpiece guide 106, as shown in FIG. 6. Preferably, pilot hole 601 has a centerline that will be substantially collinear with the desired implant shaft when workpiece guide 106 is engaged with the patient's dental arch. For example, workpiece guide 106 may be placed in a fixture that aligns workpiece guide using its fiducial references, and pilot hole 601 drilled based on the specification of the desired implant shaft in relation to the fiducial references. Pilot hole 601 may be helpful to the dental professional in starting the drilling process.

FIG. 6 also illustrates that sensors 108a-108c may be mounted on a flex circuit 602 having traces that provide power and control signals to sensors 108a-108c, and also bring sensor output signals 110a-110c out of the patient's mouth via ribbon cable 603 for communication to controller 109. Sensors 108a-108c may be encapsulated in a protective and waterproof coating. Many other mounting and signal carrying methods are possible.

In other embodiments, the outputs of sensors 108a-108c may be transmitted wirelessly, rather than through a wired connection such as flex circuit 602. In that case, a wireless transmitter such as a Bluetooth transmitter may be incorporated onto workpiece guide 106, and may receive outputs from sensors 108a-108c and relay the outputs to controller 109.

Calibration of Drill and Sensor Data.

In some embodiments, a calibration may be performed to characterize the relationship between the data provided by sensors 108a-108c and the location of drill 103. This relationship may depend on several factors, at least some of which may not be determined until the time of drilling. For example, different magnetized elements 104 may generate fields of different strengths, and there may be some variation in the pattern of magnetic flux generated by one particular magnetized element as compared with another. As drills are changed during preparation of an implant shaft, it may be necessary to recalibrate with each new drill. Additionally, the system may be used with dental handpieces of differing designs, and magnetic field 105 may be affected differently by the presence of different dental handpiece models.

FIG. 7 illustrates an example calibration station 305, according to embodiments of the invention. Calibration station 305 includes a base having a second set of sensors 701a, 701b, and 701c arranged around a hole 702. Hole 702 may have a fixed depth, so that when handpiece 101 is brought to calibration station 305 and drill 103 is inserted into hole 702 to its full depth, the distal tip of drill 103 is then in a fixed position in relationship to sensors 701a-701c. The relationship is determined by the particular design of calibration station 305. Hole 702 may be sized to permit the insertion of drill 103 with minimal play. In some embodiments, hole 702 may be fitted with a centering mechanism to accommodate drills of different sizes. Magnetized element 104 produces magnetic field 105, which is sensed by sensors 701a-701c. Sensors 701a-701c produce output signals, which may be sent via a cable 703 or another kind of interface to a computer system such as controller 109 for processing. The output signals are analyzed to characterize the shape and strength of magnetic field 105, and to characterize the spatial relationship between magnetic field 105 and drill 103. While the example shown in FIG. 7 characterizes magnetic field 105 generated by magnetized element 104 and associated with drill 103 by virtue of the relationship between magnetized element 104 and drill 103, the invention is not so limited. For example, a calibration station such as calibration station 305 may be used to characterize a magnetic field associated with drill 103 by virtue of drill 103 itself being magnetized.

In some embodiments, the sensors used in calibration station 305 may be of the same number and positioning as sensors used on workpiece guide 106. In other embodiments, more or fewer sensors may be used on calibration station 305. For example, more sensors may enable a more detailed characterization of magnetic field 105, which may enable more accurate determination of the location of drill 103 during drilling.

The characterization of the spatial relationship between magnetic field 105 and drill 103 is stored for later use.

Real-Time Display During Drilling.

Once the necessary spatial relationships have been determined, whether by design or calibration, and the characterizations stored in controller 109, controller 109 has sufficient information to compute the location of drill 103 in relation to the patient's dentition and to generate a display indicating the relationship, as shown in FIG. 1. When handpiece 101 and drill 103 are brought into proximity with sensors 108a-108c, the sensors generate outputs 110a-110c, which are read by controller 109. Controller 109 has already stored a description of the previously-characterized spatial relationship between sensors 108a-108c and the patient's dentition. For example, this relationship may be computed from the relationship of the sensors to the fiducial references of workpiece guide 106 and the relationship of the fiducial references to the patient's dentition as determined from the three-dimensional scan data. The spatial relationship of magnetic field 105 to drill 103 may have been characterized by specification or by calibration, as described above.

Controller 109 reads the sensor outputs 110a-110c and processes the outputs according to the stored relationships to determine the location of drill 103, and to produce an indication of the spatial relationship of the drill to the patient's dentition.

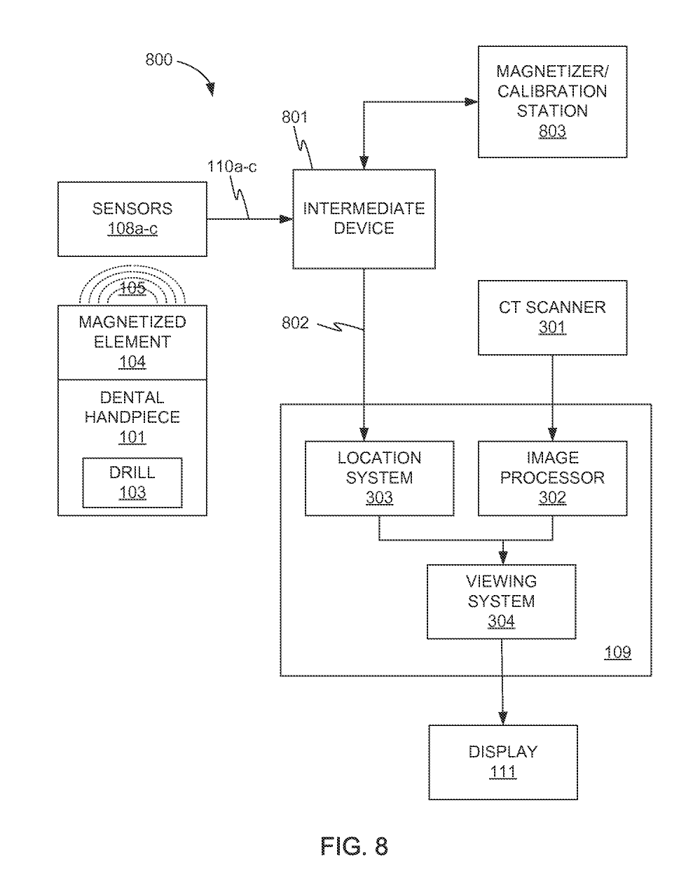

FIG. 8 is a block diagram of a system 800 in accordance with other embodiments. System 800 may include several components in common with system 300 shown in FIG. 3, and like components are given like reference numbers. In system 800, an intermediate device 801 is disposed between sensors 108a-108c and controller 109. Sensor outputs 110a-110c are communicated to intermediate device 801, rather than directly to controller 109. Intermediate device may format sensor outputs 110a-110c for transmission over an interface 802, which may be a proprietary interface, but is preferably a standard interface such as a universal serial bus (USB) interface. Intermediate device 801 may also exchange signals with a magnetizer/calibration station 803, as is described in more detail below.

Intermediate device 801 may include a microprocessor, memory, and input/output circuitry, and may thus be considered to be computerized, but in some embodiments may not include such items as a keyboard or display, and may be small enough to conveniently reside near the patient and within the reach of the implant practitioner. In this way, flexibility is provided in the placement of system components. It will be recognized that sensor outputs 110a-110c may be communicated wirelessly to intermediate device 801, and interface 802 may be a wireless interface, providing further convenience. Suitable wireless interfaces may include Bluetooth, Zigbee, IEEE 802.11, or another kind of standard or proprietary interface. In some embodiments, intermediate device 801 may serve as an electrical isolation point, for example providing galvanic isolation between controller 109 and any electronics in contact with the patient. Intermediate device 801 may also serve as a convenient connection point to separate disposable patient-contacting system components from reusable system components.

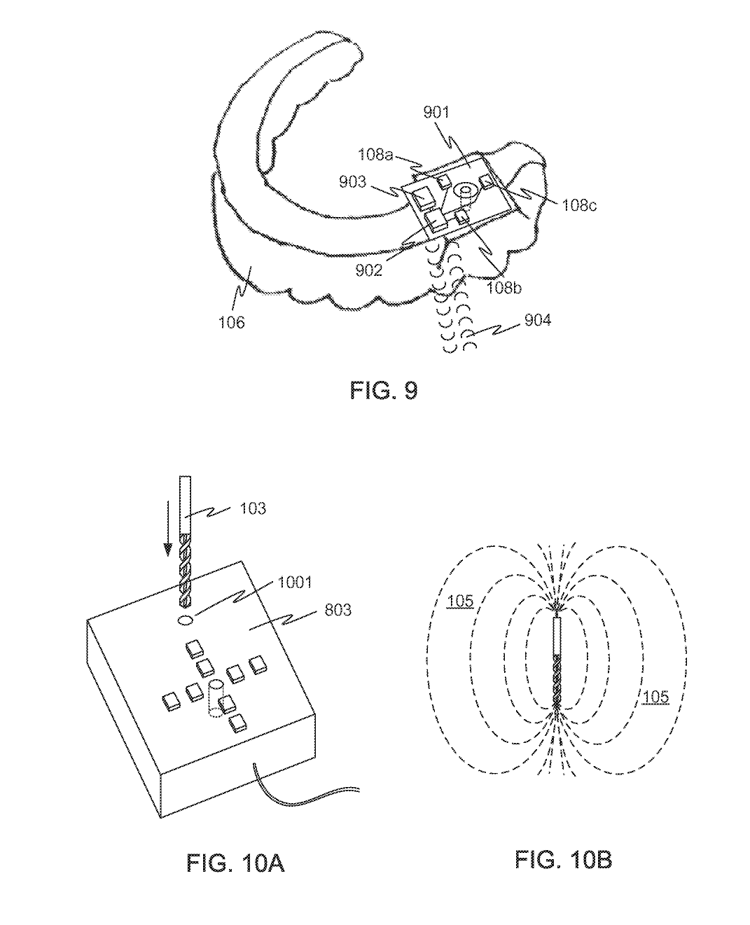

FIG. 9 illustrates an example arrangement of components that may reside in the patient's mouth when a wireless interface is used to transmit sensor outputs 110a-110c, whether to an intermediate device such as intermediate device 801, or directly to a controller such as controller 109. In the embodiment of FIG. 9, workpiece guide 106 has been prepared as described previously. Sensors 108a-108c are attached to a carrier 901, which may be a printed circuit board, flex circuit, or other suitable kind of carrier fixed to workpiece guide 106, for example by an adhesive or other suitable means. Each of sensors 108a-108c provides its outputs to circuitry 902, which may include, for example, a highly miniaturized processor system, as well as a wireless interface such as a Bluetooth interface. Power for the in-mouth circuitry may be provided by a battery 903. An antenna (not shown) may also be provided, for example as a trace on carrier 901, enabling transmission of wireless signals 904 between circuitry 902 and controller 109, intermediate device 801, or another receiver. Other power sources may be used for powering sensors 108a-108c. For example, power may be transferred to sensors 108a-108c by optical, acoustic, radio frequency, thermal, kinetic, or other means.

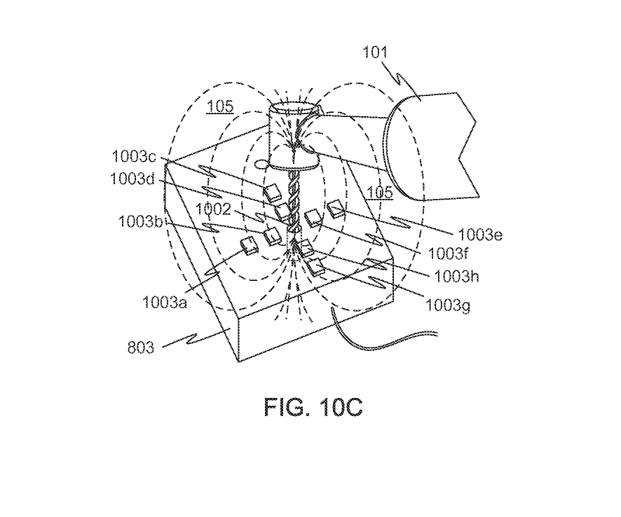

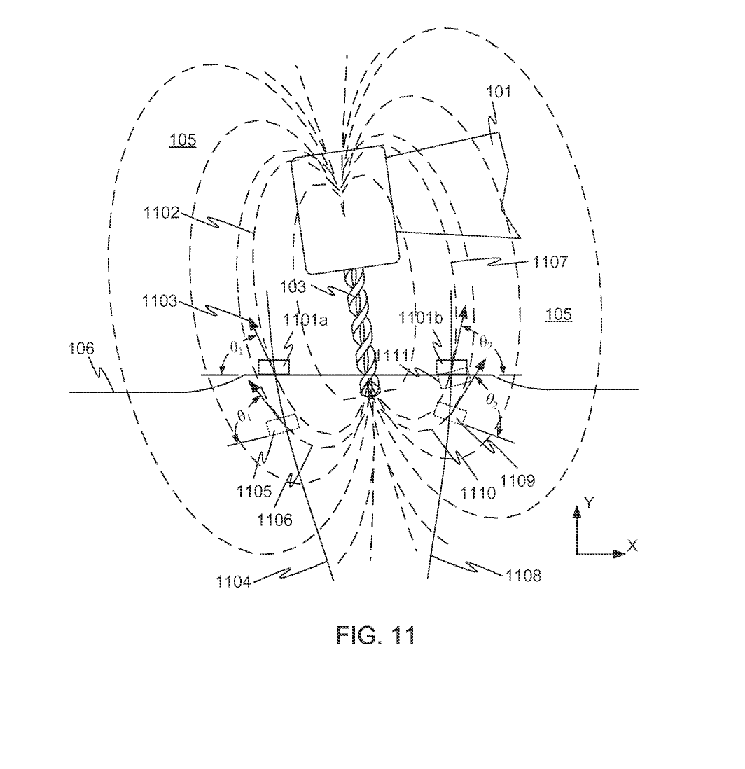

FIGS. 10A-10C illustrate an example magnetizer/calibration station 803, in accordance with embodiments. Magnetizer/calibration station 803 may be especially useful when drill 103 itself serves as the magnetized element. During an implant surgery, multiple drills may be used, for example drills of different diameters as the implant shaft enlarges. It is desirable to magnetize each drill to a known magnetization strength and pattern compatible with the system. For example, the magnetization strength should be high enough to provide robust signals from sensors 108a-108c, but low enough so that the sensors are not saturated. And because the presence of handpiece 101 may affect the magnetic field generated by a magnetized drill 103, it may be important to re-characterize the magnetic field after each drill change.

Magnetizer/calibration station 803 preferably performs both functions, although the magnetization and calibration functions could be separated and performed by different devices if desired. First, as shown in FIG. 10A, drill 103 is inserted into a receptacle 1001 in magnetizer/calibration station 803, for magnetizing drill 103. For example, a coil within magnetizer/calibration station 803 may surround drill 103 and be driven with an electric current, causing drill 103 to be magnetized. In some embodiments, drill 103 may be drawn through magnetizer/calibration station 803, for additional uniformity of magnetization. Such a system may include additional sensing means for measuring the depth of drill 103, to provide depth vs. field data. Depth information may be provided by a motion control system that controls the position of drill 103 during magnetization. In other embodiments, drill 103 may be magnetized while it is mounted in handpiece 101. The magnetization process may include demagnetizing any existing remanence from drill 103 as an initial step. FIG. 10B illustrates a representation of drill 103 after magnetization, including an approximate representation of the shape of magnetic field 105 generated by the magnetized drill 103.

After magnetization, drill 103 may be mounted to handpiece 101 and inserted into receptacle 1002 as shown in FIG. 10C. Receptacle 1002 is surrounded by a number of sensors, in this example eight sensors 1003a-1003h. More or fewer sensors may be used, the arrangement of which may or may not be co-planar. In some embodiments, drill 103 may be drawn through the plane of sensors 1003a-1003h and the sensors repeatedly read to provide strength and direction readings for magnetic field 105 at a number of positions in three-dimensional space. In other embodiments, more sensors may be provided in additional planes, so that the strength and direction of magnetic field 105 is measured in many three-dimensional positions at once. The result is a map characterizing the strength and direction of magnetic field 105. The sensor readings may be stored in a numerical array, and the array used as the characterization of magnetic field 105. In some embodiments, the sensor readings may be analyzed to create a formula describing the strength and direction of magnetic field 105 as a function of spatial position within the field. In FIG. 10C, no attempt has been made to depict the effect of handpiece 101 on the shape of magnetic field 105, but it will be recognized that the technique depicted accommodates distortion of the field caused by the presence of handpiece 101.

In some embodiments, the sensors used during drilling, such as sensors 108a-108c, may also be used for calibration. For example, when drill 103 is changed, carrier 901 may be removed from the patient's mouth and placed on a calibration station similar to magnetizer/calibration station 803, such that sensors 108a-108c are placed in a known location with respect to receptacle 1002. Drill 103 may then be passed through magnetizer/calibration station 803, and the outputs of sensors 108a-108c recorded for each of several axial locations of drill 103. The sensor outputs would be stored to provide a characterization of magnetic field 105. Once magnetic field 105 is characterized, carrier 901 would be placed back in the patient's mouth and referenced to its original location with respect to workpiece guide 106. Sensors 108a-108c would then be utilized as described above to aid in guiding the drilling process. This kind of calibration process may eliminate a potential source of error arising from differences in readings taken with different sensor sets.

FIG. 11 illustrates one example technique for determining the location of drill 103 with respect to sensors 1101a and 1101b, and thus with respect to the patient's dentition. The example of FIG. 11 depicts only two dimensions for ease of explanation, but it will be recognized that the technique may be generalized to a three-dimensional system. In FIG. 11, handpiece 101 and drill 103 are shown in a particular location with respect to sensors 1101a and 1101b, which are fixed to workpiece guide 106. This example utilizes drill 103 as the magnetized element. A particular flux line 1102 of magnetic field 105 passes through sensor 1101a, at an incident angle θ₁, and with a strength represented by the length of vector 1103. The output of sensor 1101a indicates the field strength and direction of magnetic field 105 as seen by sensor 1101a—that is the output indicates the field strength and θ₁.

The output of sensor 1101a alone is not sufficient to characterize the location of sensor 1101a within magnetic field 105. For example, sensor 1101a could be at any position along isomagnetic locus 1104, which is the locus of all points within magnetic field 105 having the same magnetic field strength as the point at which sensor 1101a happens to reside. (Only portions of the isomagnetic loci in FIG. 11 are illustrated. In practice, each isomagnetic locus will be a closed curve.) Given the field strength reading from sensor 1101a, isomagnetic locus 1104 may be determined from the previous characterization of magnetic field 105, for example by interpolating within a numerical array describing the field, or formulaically if the field has been described by a mathematical formula. Another possible location of sensor 1101a within magnetic field 105 is shown at location 1105. If sensor 1101a and magnetic field 105 were in a relationship that placed sensor 1101a at location 1105, at an angle of θ₁ with respect to field line 1106, sensor 1101a would give an identical output. More information is needed to determine the relationship of magnetic field 105 to the sensors.

Similarly, sensor 1101b is crossed by field line 1107 at an angle θ₂. Thus, the system can determine that sensor 1101b is located somewhere on isomagnetic locus 1108, but given only the output of sensor 1101b, cannot determine where on isomagnetic locus 1108. For example, sensor 1101b could be at location 1109, oriented at an angle of θ₂ with respect to field line 1110.

By combining the information from both sensor outputs with previously determined information about the orientation of sensors 1101a and 1101b, it is possible to uniquely determine the locations of sensors 1101a and 1101b within magnetic field 105. In some embodiments, it is known how far apart sensors 1101a and 1101b actually are on workpiece guide 106. Given that information and a hypothetical location of one sensor, it is possible to calculate the expected position of the other sensor, and test whether the two locations fit the measured data. For example, if it is assumed that sensor 1101a is at location 1105, then sensor 1101b would be expected to be at location 1111. While position 1111 is quite close to the actual X-Y position of sensor 1101b, hypothetical location 1111 is oriented incorrectly with respect to the local field lines, and cannot be the actual position of sensor 1101b. Thus, location 1105 cannot be the correct location of sensor 1101a. Potential locations for sensor 1101a along isomagnetic locus 1104 may be searched until the predicted location of sensor 1101b matches the actual angular data from sensor 1101b. Once a matching pair of locations is found, the locations of sensors 1101a and 1101b within magnetic field 105 is ascertained. From that information, it is straightforward to calculate the orientation of magnetic field 105 with respect to workpiece guide 106, and accordingly with respect to the patient's dentition. And because the location of drill 103 is known with respect to magnetic field 105, the location of drill 103 can be calculated with respect to the patient's dentition. From that relationship and the previously-stored radiographic image, the system can generate the display graphically illustrating the location of drill 103 with respect to the patient's dentition. Similarly, because the location of the desired implant shaft is also known, the system can generate the indication of the location of drill 103 with respect to the desired implant shaft.

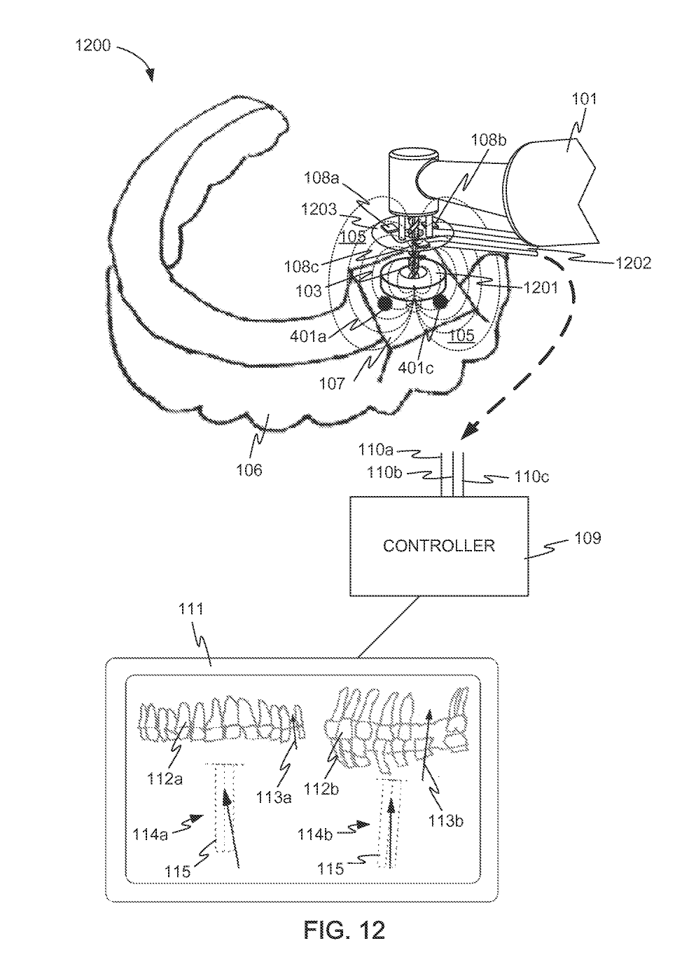

FIG. 12 illustrates a system 1200 in accordance with another embodiment of the invention, for indicating the location of a dental drill. System 1200 includes some components similar to components shown in FIG. 1, and like components are given like reference numbers. In the system of FIG. 1, magnetic element 104 is fixed to drill 103, and sensors 108a-108c are fixed to workpiece guide 106. System 1200 reverses that arrangement.

In system 1200, a magnetized element 1201 is fixed to workpiece guide 106, and generates a magnetic field 105. Sensors 108a-108c are fixed in relation to handpiece 101, and consequently in relation to drill 103. As handpiece 101 is moved, sensors 108a-108c are exposed to different parts of magnetic field 105, and produce different outputs 110a-110c. Sensor outputs 110a-110c are provided to controller 109, for example via a flexible cable 1202 (shown in only a partial view), or via a wireless connection. An intermediate device similar to intermediate device 801 may also be present. Controller 109 processes sensor outputs 110a-110c to provide an indication of the location of drill 103 in relation to the dentition of a patient wearing workpiece guide 106. For example, the strength and shape of magnetic field 105 and its spatial relationship to the patient's dentition may be characterized, and the spatial relationship of sensors 108a-108c to drill 103 may be characterized, and this information supplied to controller 109, which then processes sensor outputs 110a-110c according to these previously-characterized relationships to determine the location of drill 103 with respect to the patient's dentition. As in the embodiments described above, location may be determined by interpolating within a numerical array describing the field, or formulaically if the field has been described by a mathematical formula.

To characterize the relationship between magnetic field 105 and the patient's dentition, the relationship of magnetic field 105 to magnetized element 1201 may first be characterized. For example, a set of sensors similar to those on calibration station 305 or magnetizer/calibration station 803 may be used. Magnetized element 1201 may be placed in a known relationship to the sensors, and readings produced by the sensors used to characterize magnetic field 105. In other embodiments, magnetized element 1201 may be supplied from the factory with a data file describing magnetic field 105.

Magnetized element 1201 may then be placed in a known location with respect to workpiece guide 106 (whose relationship to the patient's dentition is known from the process of fabricating workpiece guide 106). For example, surface 107 may be a planar surface coincident with the plane defined by radiopaque fiducial markers 401a-401c. A pilot hole 601 is formed in workpiece guide, a pin (which may preferably be a stepped pin) may be placed in pilot hole 601 and magnetized element 1201 slipped over the pin until magnetized element 1201 touches surface 107 of workpiece guide 106. Magnetized element 1201 may then be fixed to workpiece guide 106, for example using an epoxy or other adhesive. This process completely defines the location of magnetized element 1201 with respect to the patient's dentition (once workpiece guide 106 is replaced in the patient's mouth).

The relationship of sensors 108a-108c to drill 103 may be characterized by mechanically positioning drill at a predetermined location with respect to sensors 108a-108c. For example, a fixture may be utilized to set the depth of insertion of drill 103 into handpiece 101 such that the distance from the bottom of sensor mounting plate 1203 to the tip of drill 103 is set consistently to a predetermined value, even when drill 103 is changed during the implant procedure. In other embodiments, a calibration fixture having a previously-characterized magnetic field could be used.

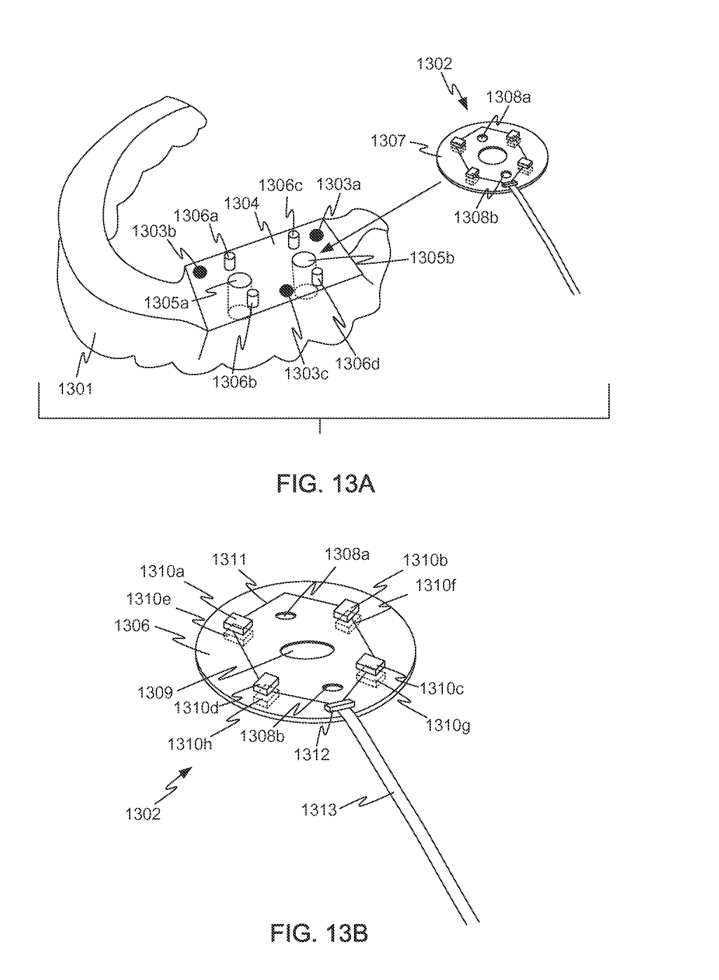

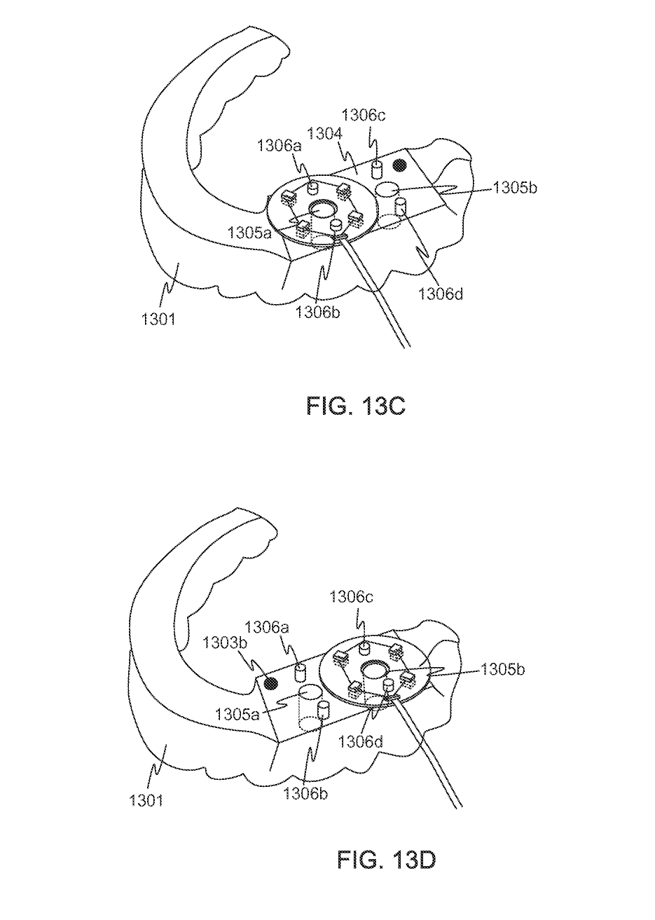

FIG. 13A illustrates a workpiece guide 1301 and a sensor assembly 1302, in accordance with embodiments of the invention. Workpiece guide 1301 and sensor assembly 1302 are adapted for performing two implants in a single treatment session, although it will be recognized that certain features of the system are applicable to single-implant embodiments, or to embodiments adapted for three or more implants.

Example workpiece guide 1301 is configured for performing implants at two adjacent tooth locations. Using the techniques described previously, a dental professional has selected the locations of two implant shafts. Workpiece guide 1301 has been fabricated to conform to the patient's dentition, and includes three fiducial markers 1303a-1303c affixed to surface 1304. Two pilot holes 1305a and 1305b have been formed in workpiece guide 1301, preferably aligned with the two desired implant shafts. While workpiece guide 1301 is configured for performing two implants, it will be recognized that in other embodiments a workpiece guide may be configured for performing more implants, including implants at non-adjacent tooth locations. Also, a different number of fiducial markers could be used. For example, each implant site could use its own respective set of fiducial markers.

Also positioned near each pilot hole 1305a, 1305b is a set of alignment pins. For example, alignment pins 1306a and 1306b are positioned near pilot hole 1305a, and alignment pins 1306c and 1306d are positioned near pilot hole 1305b. The alignment pins may be placed in known relationship to the other features of workpiece guide 1301. For example, at the time pilot holes 1305a and 1305b are formed, holes for receiving alignment pins 1306a-1306d may be formed. Alignment pins 1306a-1306d can then be inserted into the prepared holes, for example by press fitting. Alignment pins 1306a-1306d may be made of any suitable material, but may preferably be made of a polymer such as polycarbonate or acrylonitrile butadiene styrene (ABS), a non-magnetic metal such as titanium, or another material that will have little or no effect on magnetic fields in the area.

Example sensor assembly 1302 includes a circuit board 1307 having alignment holes 1308a and 1308b, spaced for engagement with the respective sets of alignment pins 1306a-1306d. Thus, sensor assembly 1302 can be engaged with a first set of alignment pins to aid in drilling an implant shaft for a first implant, and then moved to engage a different set of alignment pins for drilling a different implant shaft for a second implant. For example, sensor assembly 1302 may be engaged with alignment pins 1306a and 1306b for assisting in drilling an implant shaft associated with pilot hole 1305a, and then moved to engage with alignment pins 1306c and 1306d for assisting in drilling an implant shaft associated with pilot hole 1305b.

FIG. 13B shows example sensor assembly 1302 in more detail. Circuit board 1307 may be a double sided printed circuit board or flex circuit or another kind of circuit carrier, and may have multiple layers. Besides alignment holes 1308a and 1308b, circuit board 1307 includes a clearance opening 1309, allowing clearance for a drill to reach the appropriate pilot hole. Circuit board 1307 also carries eight sensors 1310a-1310h in this example. Four sensors 1310a-1310d are mounted on the top surface of circuit board 1306, and four additional sensors 1310e-1310h (shown in broken lines) are mounted to the bottom surface of circuit board 1307. While sensors 1310e-1310h are shown as being mounted directly below sensors 1310a-1310d, this is not a requirement. The sensors also need not be mounted symmetrically around clearance opening 1309. This “dual quad” arrangement having two layers of four sensors each may provide improved accuracy in determining the position of a drill as compared with a single-layer arrangement of sensors. Signals from sensors 1310a-1310h are carried by traces 1311 in circuit board 1307 (the traces are shown in simplified form) to a connector 1312, and then to a cable 1313 for communicating the signals to a controller such as controller 109, or to an intermediate device such as intermediate device 801.

Many different variations and system architectures are possible. For example, if a flex circuit is used, no connector 1312 may be necessary. Or in a wireless arrangement similar to the arrangement of FIG. 9, no cable 1313 may be necessary. In other embodiments, different numbers of sensors may be used. For example, a “dual triad” arrangement may be used, with three sensors on top of circuit board 1307 and three sensors on the bottom side of circuit board 1307.

FIG. 13C shows sensor assembly 1302 engaged with alignment pins 1306a and 1306b, for aiding in drilling an implant shaft associated with pilot hole 1305a. Alignment pins 1306a and 1306b assist in holding sensor assembly 1302 in a first fixed position in relation to workpiece guide 1301. Many other alignment mechanisms may be envisioned for enabling a sensor assembly such as sensor assembly 1302 to be moved from one implant location to another. For example, a sleeve could be placed in each pilot hole and the sensor assembly aligned with the sleeve to center over the pilot hole. Or a raised shape may be formed in workpiece guide 1301 near each pilot hole and clearance opening 1309 of sensor assembly 1302 placed over the raised shape to register sensor assembly 1302 to workpiece guide 1301. The raised shape may have a polygonal shape, for example square or trapezoidal, and clearance opening 1309 may have a complementary shape, to prevent rotation of sensor assembly 1302. By disengaging sensor assembly 1302 from alignment pins 1306a and 1306b, sensor assembly 1302 can be moved to a second fixed position with respect to workpiece guide 1301. FIG. 13D shows sensor assembly 1302 engaged with alignment pins 1306c and 1306d, for aiding in drilling an implant shaft associated with pilot hole 1306a.

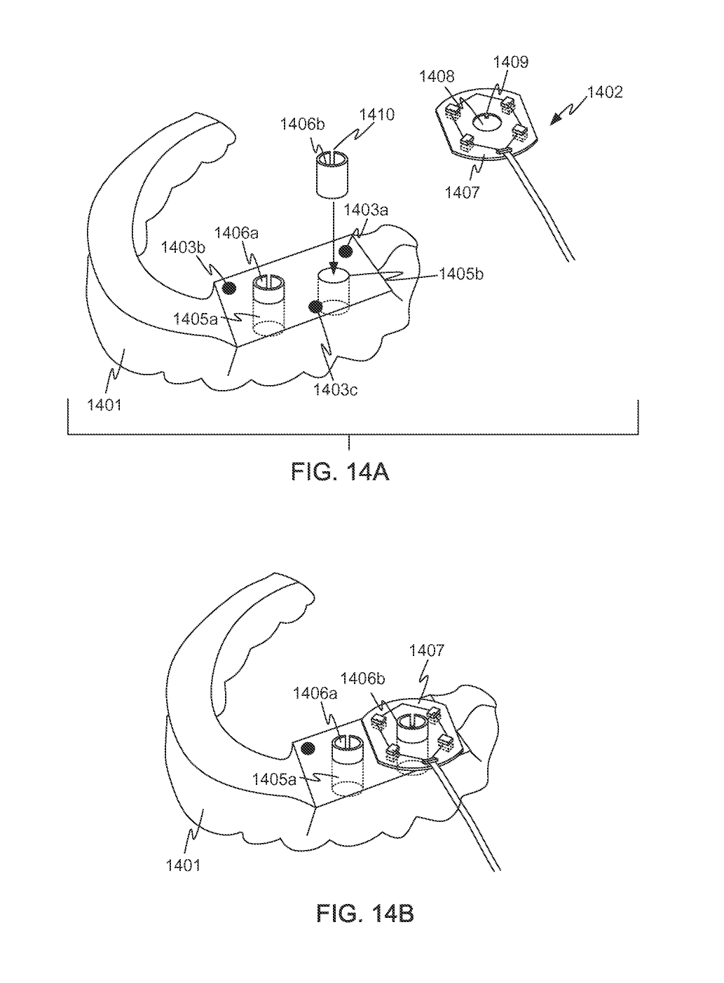

FIG. 14A illustrates a workpiece guide 1401 and a sensor assembly 1402, in accordance with other embodiments of the invention. Workpiece guide 1401 and sensor assembly 1402 are adapted for performing two implants in a single treatment session, although it will be recognized that certain features of the system are applicable to single-implant embodiments, or to embodiments adapted for performing three or more implants. Using workpiece guide 1401 in a manner similar to that described above, pilot holes 1405a and 1405b may be placed in line or approximately in line with desired implant shafts previously specified by the dental professional. Fiducial markers 1403a-1403c may be used in the process of determining the positions of pilot holes 1405a and 1405b. Sleeves 1406a and 1406b are placed in pilot holes 1405a and 1405b. Sleeves 1406a and 1406b are preferably made of a suitable radiopaque, non-magnetic material such as an acrylic doped with barium sulfate. Circuit board 1407 of sensor assembly 1402 includes an alignment hole 1408 sized to fit snugly over one of sleeves 1406a or 1406b. A tab 1409 is sized to fit within a gap or keyway 1410 formed in each sleeve.

FIG. 14B shows sensor assembly 1402 in place over workpiece guide 1401. Tab 1409 prevents rotation of sensor assembly 1402 about the axis of the sleeve with which it is engaged. The positions of the sleeve keyways may be determined with a second radiographic characterization of workpiece guide 1401, or by other means. Alternatively, sleeves 1406a and 1406b may be placed in approximate locations before the radiographic characterization of workpiece guide 1401 in the patient's mouth, and may serve as fiducial references instead of or in addition to fiducial markers 1403a-1403c.

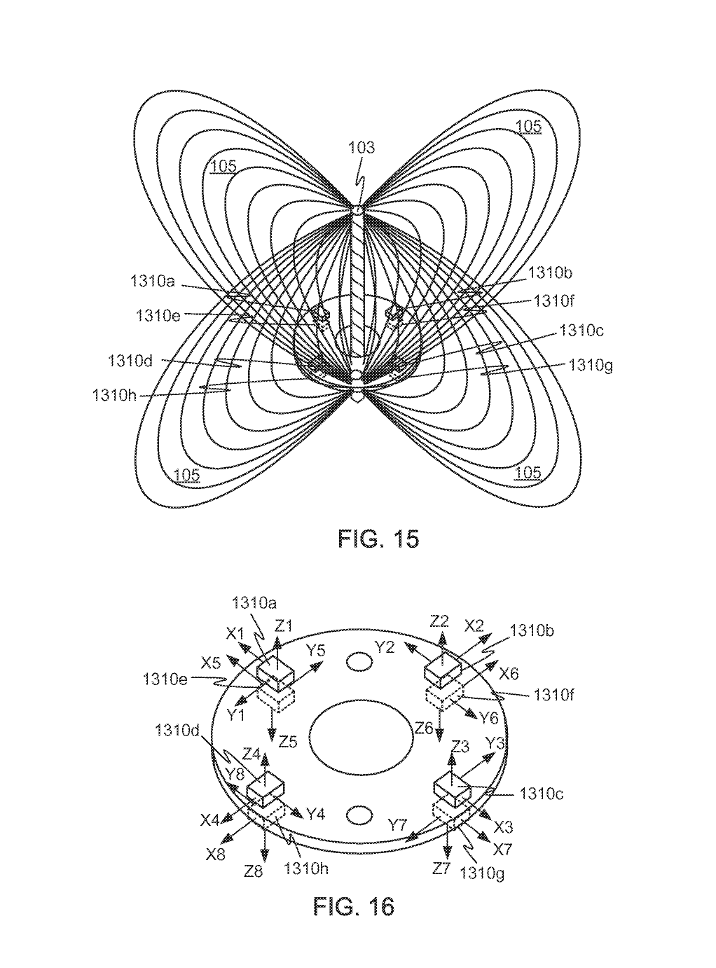

FIGS. 15-19 illustrate certain component relationships and an alternate technique for determining the position of drill 103 in relation to the patient's dentition, in embodiments of the invention. For example, FIG. 15 shows the relationship of example magnetic field 105 with sensors in a “dual quad” arrangement, such as sensors 1310a-1310h, in accordance with example embodiments. Magnetic field 105 is represented in FIG. 15 by four lobes, but it will be recognized that in this example, magnetic field 105 may be generally rotationally symmetric about the axis of drill 103. It is assumed that sensors 1310a-1310h have been placed in a known fixed relationship with the patient's dentition, for example using the techniques described above. In other embodiments, magnetic field 105 may not be rotationally symmetric, for example if the body of a handpiece holding the drill affects the field significantly.

FIG. 16 illustrates a coordinate system useful in describing the behavior of sensors. In this example, each sensor 1310a-1310h is a model HMC5883L 3-Axis Digital Compass integrated circuit available from Honeywell International Inc., and has its own local coordinate system (Xₙ,Yₙ,Zₙ), while the overall system is conveniently described using radial coordinates (Z,R, Φ). Each sensor of this type produces three outputs, indicating the strength of the magnetic field in each of the three coordinate axes.

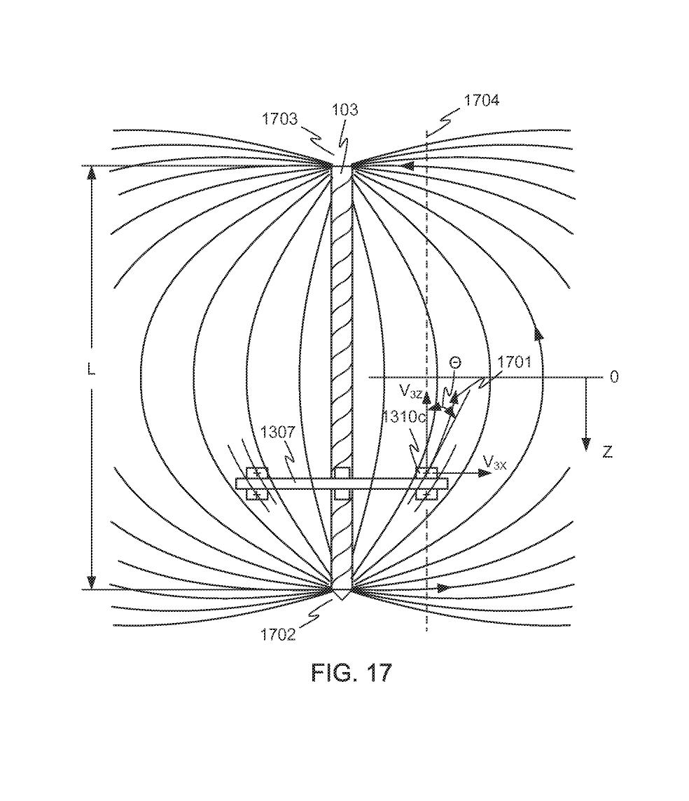

FIG. 17 illustrates an orthogonal view of the interaction of field 105 with the sensors in more detail. Using sensor 1310c as an example, in the position shown, a particular flux line 1701 passes through the measurement location of sensor 1310c, at an angle of Θ. The angle Θ can be determined from the sensor outputs as a tan(V_{3X}/V_{3Z})*180/π. If circuit board 1307 were to be positioned at Z=0, it can be seen that the flux lines are nearly vertical, so the angle Θ would be essentially 0 degrees. At bottom end 1702 of drill 103, the flux lines emanate nearly horizontally from drill 103, so if circuit board 1307 were to be positioned at the bottom of drill 103 (but still held horizontal as shown), the angle Θ would be about 90 degrees. At top end 1703 of drill 103, the flux lines converge nearly horizontally toward drill 103, so if circuit board 1307 were to be positioned at the top of drill 103 (but still held horizontal as shown), the angle Θ would be about −90 degrees.

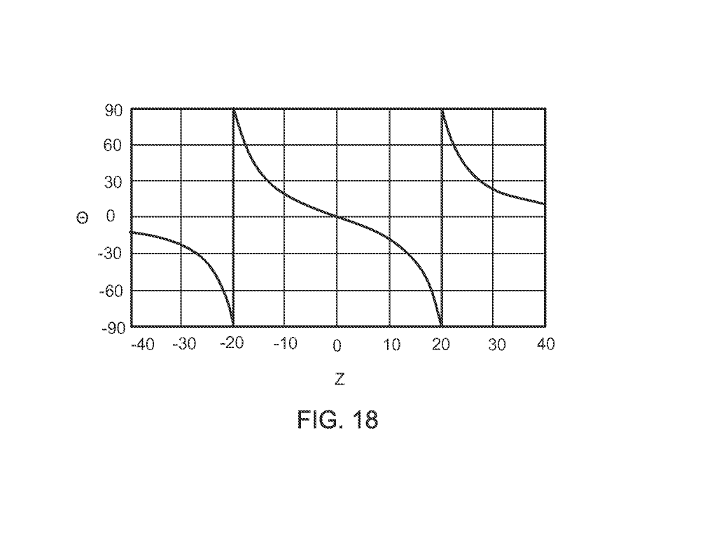

FIG. 18 shows an approximate representation of angle Θ as a function of position along the Z direction (as sensor 1310c traverses dashed path 1704), for a drill having a length L=40 mm. The exact relationship of angle Θ to Z position will depend on the particular field shape, but for a magnetized drill, may generally be a monotonic function over much of the length of drill 103. For the simple case where the drill is centered among the sensors and perpendicular to the plane of the sensors, the drill depth could be determined from the angle Θ measured at any one of the sensors.

However, it may be desirable to average the readings of the sensors, to reduce noise and to at least partially cancel the effects of tilt and de-centering of the drill. For example, de-centering of the drill within the sensor constellation will tend to reduce the angles Θ measured by sensors toward which the drill is moved, and will tend to increase the angles Θ measured by sensors away from which the drill is moved. Similarly, tilt of the drill will tend to increase the angles measured on one side of the drill and reduce the angles measured on the other side of the drill. By averaging the angles Θ measured at all of the sensors (eight sensors in the example of FIGS. 15-17), these effects are at least approximately canceled, and a reasonably accurate estimate of drill depth can be obtained from a calibration curve similar to FIG. 18. It will be recognized that the readings from the sensors on the bottom of the circuit board may require a sign reversal before averaging.

The depth estimate obtained in this way may greatly simplify the remaining determination of drill location as a function of the sensor readings. Once the depth is approximately known, the required range of search within the calibration data may be greatly reduced, as compared with trying to locate the drill from an arbitrary set of sensor readings.

It has also been observed that the portion of the calibration curve of FIG. 18 corresponding to the length of the drill (−20 to +20 in the example of FIG. 18) can be substantially linearized by multiplying the ratio of the sensor outputs by a constant prior to applying the arctangent function. That is, a plot of a tan(k*V_{3X}/V_{3Z})*180/π will be nearly a straight line in the region of interest, for an appropriate value of k. The value of k will depend on the particular system geometry and other implementation-specific factors, and can be easily chosen by plotting the calibration curve with different values of k until a nearly-linear curve is obtained. A linearized calibration curve may further simplify the determination of drill location.

Another aspect that may simplify the determination of drill location is that during drilling, circuit board 1307 will be positioned between the ends of drill 103. Thus only the monotonic range of a calibration curve similar to FIG. 18 need be considered. In FIG. 18, the monotonic range includes values of Z from about −20 to about +20. The dental professional may assure that location estimation begins only after the end of the drill has passed through circuit board 1307, for example by signaling to the system that the drill has been inserted into the appropriate pilot hole. In some embodiments, the starting of the drill may signal to the system that location estimation is to begin, and the dental professional may simply wait until the drill is positioned within the pilot hole before starting the drill.

Once the drill depth has been estimated, other relationships in sensor output may be exploited to further refine the estimate of drill location. For example, translation of drill 103 may cause sensors toward which drill 103 is moved to register stronger field readings than sensors from which drill 103 is moved away. Similarly, tilt of drill 103 may cause some sensors to read steeper field angles and other sensors to read field angles that are less steep. Non-zero readings of field components in the Y directions of the sensors indicate that the drill is angled.

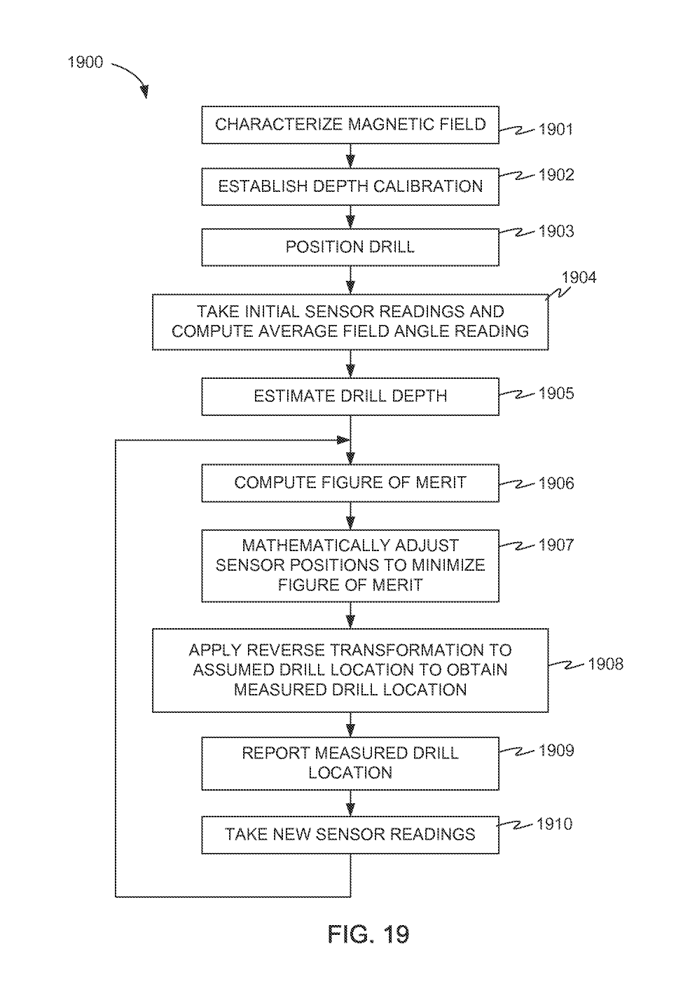

Techniques such as these may be combined into a method of establishing the drill location from the sensor readings. FIG. 19 is a flowchart of a method 1900 according to one example embodiment. In step 1901, the magnetic field is characterized, for example using a fixture and methods as described above in relation to FIGS. 10A-10C. The characterization of the magnetic field may take the form of a table of measured sensor values at different locations within the field. In other embodiments, the sensor values may be fit to a formulaic description of the field, from which field strengths and angles can be computed as a function of location within the field.

In step 1902, a depth calibration curve is established. For example, the depth calibration curve may be similar to the curve shown in FIG. 18, showing the field angle measured by a sensor when the drill is centered within the sensor constellation. The depth calibration curve may be based on average readings taken by multiple sensors during the calibration process. In step 1903, the drill is placed in position for drilling, with the circuit board holding the sensors positioned between the ends of the drill. In step 1904, an initial set of sensor readings is taken, and an average field angle reading is computed. It will be recognized that the readings from sensors on the bottom of the circuit board may be reversed in sign before the averaging.

In step 1905, the average field angle reading is used to determine an initial depth estimate from the depth calibration curve. This estimate assumes that the drill is perpendicular to the average plane of the sensors, and is centered within the constellation of sensors. In step 1906, a figure of merit is computed, indicating how well the assumed position of the sensors agrees with the initial sensor readings. For example, predicted sensor readings may be computed based on the assumed positions of the sensors within the characterized magnetic field, and compared with the actual initial sensor readings. The figure of merit could be, for example, the sum of the squares of the differences between the respective predicted and actual sensor readings, although other figures of merit may be envisioned. For example, absolute value differences could be summed, different sensor readings could be weighted differently, or other variations may be used. When eight sensors are used, each producing three readings, the computation of the figure of merit may include up to 24 differences between predicted and actual readings. In some embodiments, the estimation of drill position may be performed using multiple subsets of the sensors, so that if the estimates disagree, it may be assumed that an error has occurred, and drilling can be stopped.

In step 1907, the positions of the sensors are mathematically adjusted to minimize the figure of merit. For example, the assumed position of circuit board 1307, and consequently the assumed positions of sensors 1310a-1310h, may be mathematically moved to a new location in space. The movement may include translation in depth, two lateral translations (perpendicular to drill 103), and rotations in at least two degrees of freedom having rotational axes in the sensor plane, for a total of up to five degrees of freedom. If it is assumed that the magnetic field is not rotationally symmetric, the movement may also include rotation around the longitudinal axis of drill 103 as well, resulting in six degrees of freedom. It will be recognized that step 1907 is highly simplified in FIG. 19, and may involve many trial mathematical positionings of the sensors and computations of the figure of merit at each trial position. Any suitable mathematical technique may be used, for example a gradient descent algorithm, the simplex algorithm, or another optimization algorithm. Once the figure of merit is minimized, the relationship of the sensors and the magnetic field is known. That is, the transformation required for the assumed sensor positions to produce predicted sensor readings that agree with the actual sensor readings is known. The reverse of this transformation is applied to the assumed drill position in step 1908, and the resulting measured drill location is reported in step 1909. The measured drill location may then be used to construct a display such as the display shown in FIG. 1 or FIG. 12, showing the measured position of the drill in relation to the patient's dentition, a desired implant shaft, or both.

Some of the steps of method 1900 may then be repeated, so that the display can be updated, preferably substantially in real time. For example, a new set of sensor readings is taken in step 1910, and control may be passed to step 1906 for a new computation of the figure of merit, and a new evaluation of the drill location.

Many variations are possible. For example, in other numerical embodiments, the location of the magnetic field may be mathematically perturbed rather than the locations of the sensors. In other embodiments, where the magnetic field has been characterized using a formula, it may be possible to backsolve the formula to obtain the location of the drill. It is to be understood that all workable combinations of the features and element disclosed herein are also considered to be disclosed.

The embodiments disclosed above are exemplary and are not to be construed as limiting the scope of the invention. Many variations of the methods and devices described herein are available to the skilled artisan without departing from the scope of the invention.

### EMBODIMENTS

### Embodiment 1

A system for indicating the location of a dental drill, the system comprising: a dental handpiece comprising the dental drill; and a plurality of sensors that detect a magnetic field and produce a set of respective sensor outputs, the sensor outputs usable at least in part to indicate the location of the dental drill.

### Embodiment 2

The system of embodiment 1, further comprising a magnetic element that is fixed in relation to the dental drill and generates the magnetic field.

### Embodiment 3

The system of embodiment 1, wherein the dental drill is magnetized and generates the magnetic field.

### Embodiment 4

The system of embodiment 1, further comprising a magnetic element that is fixed in relation to the dentition of a patient, and wherein the sensors are fixed in relation to the dental handpiece.

### Embodiment 5

The system of any one of the embodiments 1 to 3, further comprising a workpiece guide registered to a patient's dentition, wherein the sensors are fixed in relation to the workpiece guide.

### Embodiment 6

The system of embodiment 5, wherein the sensors are movable from a first fixed position in relation to the workpiece guide to a second fixed position in relation to the workpiece guide.

### Embodiment 7

The system of any one of the embodiments 1 to 6, further comprising a carrier on which the sensors are mounted, at least three of the sensors mounted to a first surface of the carrier, and at least three of the sensors mounted to a second surface of the carrier.

### Embodiment 8

The system of embodiment 7, wherein four of the sensors are mounted to a first surface of the carrier, and four of the sensors are mounted to a second surface of the carrier.

### Embodiment 9

The system of any one of the embodiments 1 to 8, further comprising a controller that receives the sensor outputs and processes the outputs to produce an indication of the spatial relationship of the dental drill to a patient's dentition.

### Embodiment 10

The system of embodiment 9, wherein the controller processes the sensor outputs according to a spatial relationship between the sensors and the patient's dentition and according to a spatial relationship between the magnetic field and the dental drill.

### Embodiment 11

The system of any one of the embodiments 9-10, further comprising an intermediate device that receives the sensor outputs and relays the sensor outputs to the controller.

### Embodiment 12

The system of any one of the embodiments 9-11, further comprising a wireless interface by which the sensor outputs are transmitted to reach the controller.

### Embodiment 13

The system of any one of the embodiments 9-12, wherein the controller repeatedly updates the indication of the spatial relationship of the dental drill to the patient's dentition, substantially in real time.

### Embodiment 14

The system of any one of the embodiments 1-13, further comprising an electronic display, and wherein the indication of the spatial relationship of the dental drill to the patient's dentition is pictorially represented on the electronic display.

### Embodiment 15

The system of any one of the embodiments 1-14, wherein the indication of the spatial relationship of the dental drill to the patient's dentition comprises: a pictorial representation of the patient's dentition; and a representation of the location of the dental drill location superimposed on the pictorial representation of the patient's dentition.

### Embodiment 16

The system of any one of the embodiments 14-15, wherein the pictorial representation of the patient's dentition is derived from a radiographic image of the patient's dentition.

### Embodiment 17

The system of any one of the embodiments 14-16, wherein the pictorial representation of the patient's dentition is a representation of a three-dimensional model of the patient's dentition.

### Embodiment 18

The system of any one of the embodiments 9-17, wherein the controller further produces an indication of the spatial relationship of the dental drill to a previously-specified implant shaft within the patient's dentition.

### Embodiment 19

The system of embodiment 18, wherein the controller further produces a warning signal when the dental drill departs from the previously-specified implant shaft by at least a predetermined amount.

### Embodiment 20

The system of any one of the embodiments 1-19, further comprising a calibration station that further includes: a receptacle for the dental drill; and a second plurality of sensors fixed in relation to the receptacle, each of the second plurality of sensors producing an output, and wherein the outputs of the second plurality of sensors are usable to characterize the spatial relationship of the magnetic field to the dental drill when the dental drill is placed in the receptacle.

### Embodiment 21

A method of indicating the location of a dental drill, the method comprising: reading outputs produced by a set of sensors, wherein the sensors detect a magnetic field, and wherein the sensor outputs are usable to detect the location of a dental drill in relation to the sensors; processing the sensor outputs to produce an indication of the spatial relationship of the dental drill to a patient's dentition; and displaying the indication of the spatial relationship of the dental drill to the patient's dentition.

### Embodiment 22

The method of embodiment 21, wherein processing the outputs comprises processing the outputs according to a spatial relationship between the sensors and the patient's dentition and according to a spatial relationship between the magnetic field and the dental drill.

### Embodiment 23

The method of any one of the embodiments 21-22, wherein displaying an indication of the spatial relationship of the dental drill to the patient's dentition comprises repeatedly updating the display of the indication of the spatial relationship of the dental drill to the patient's dentition, substantially in real time.

### Embodiment 24

The method of any one of the embodiments 21-23, wherein reading the outputs of a set of sensors comprises reading the outputs of the sensors via a wireless interface.

### Embodiment 25

The method of any one of the embodiments 21-24, further comprising, indicating on the display the location of the dental drill in relation to a previously-specified implant shaft.

### Embodiment 26

The method of any one of the embodiments 21-25, further comprising: comparing the location of the dental drill with the previously-specified implant shaft; and producing a warning signal when the dental drill departs from the previously-specified implant shaft by at least a predetermined amount.

### Embodiment 27

The method of embodiment 26, wherein the warning signal comprises one or more signals selected from the group consisting of a visual cue and a sound cue, alone or in any combination.

### Embodiment 28

A workpiece guide, comprising: a dental arch portion that conforms to the dentition of a particular patient; and a set of sensors fixed in relation to the dental arch portion, each sensor capable of producing an output that indicates at least one characteristic of a magnetic field.

### Embodiment 29

The workpiece guide of embodiment 28, wherein the dental arch portion defines a pilot hole located, when the workpiece guide is engaged with the dental arch of the particular patient, substantially at the centerline of a desired implant shaft.

### Embodiment 30

The workpiece guide of any one of the embodiments 28-29, further comprising at least three non-collinear radiopaque fiducial markers on the workpiece guide.

### Embodiment 31

The workpiece guide of any one of the embodiments 28-30, wherein the sensors are movable from a first fixed position in relation to the workpiece guide to a second fixed position in relation to the workpiece guide.

### Embodiment 32

A method, comprising: fabricating a workpiece guide of a configuration to engage the dentition of a particular patient having an implant site; placing a set of fiducial references on the workpiece guide; and fixing a sensor to the workpiece guide, the sensor capable of, when the sensor is exposed to a magnetic field, producing an output indicating an aspect of the magnetic field.

### Embodiment 33

The method of embodiment 32, further comprising: engaging the workpiece guide with the dental arch of the patient; obtaining a radiographic image of the workpiece guide and the patient's dental arch, the radiographic image depicting the fiducial references; determining from the radiographic image the location of a desired implant shaft for placing an implant at the implant site; and characterizing the location of the desired implant shaft with respect to the locations of the fiducial references.

### Embodiment 34

The method of embodiment 33, further comprising: forming a pilot hole in the radiographic workpiece guide, wherein the centerline of the pilot hole will be substantially collinear with the centerline of the implant shaft when the radiographic workpiece guide is engaged with the patient's dental arch.

### Embodiment 35

The method of any one of the embodiments 33-34, further comprising: bringing a dental handpiece comprising a dental drill into proximity with the sensor, wherein an element fixed to the handpiece produces a magnetic field, such that the sensor detects the magnetic field; obtaining an output from the sensor; processing the sensor output to determine the spatial relationship between the dental drill and the patients' dentition; and displaying, on a visual display, an indication of the spatial relationship of the dental drill to the patient's dentition.

### Embodiment 36

The method of embodiment 35, further comprising calibrating the spatial relationship between the magnetic field and the dental drill.

### Embodiment 37

The method of any one of the embodiments 33-36, further comprising simultaneously displaying, on the visual display, an indication of the spatial relationship of the dental drill to the desired implant shaft.

### Embodiment 38

The method of embodiment 37, further comprising producing a warning signal when the dental drill departs from the previously-specified implant shaft by at least a predetermined amount.

### Embodiment 39

A computerized controller, comprising: an image processor that receives a radiographic image of a patient's dentition; a location system that receives outputs from one or more sensors, wherein the sensors detect at least one aspect of a magnetic field, and the sensor outputs change as the spatial relationship of the magnetic field and the sensors changes due to changes in the location of a dental handpiece that includes a dental drill, and wherein the location system processes the sensor outputs to determine the location of the dental drill in relation to the patient's dentition; and a viewing system that generates a display image at a computer display such that the generated display image comprises an image of the patient's dentition and a depiction of the location of the dental drill relative to the patient's dentition as determined by the location system.

### Embodiment 40

The computerized controller of embodiment 39, wherein the location system receives updated sensor outputs and determines based at least in part on the updated sensor outputs an updated location of the handpiece in relation to the patient's dentition, and the viewing system adjusts the generated display image to show the updated location of the dental drill relative to the patient's dentition.

### Embodiment 41

The computerized controller of any one of the embodiments 39-40 wherein the generated display image further comprises a depiction of the location of the dental drill relative to a desired implant shaft.

### Embodiment 42

The computerized controller of any one of the embodiments 39-41, further comprising a computer processor that performs operations of the location system and image processor.

### Embodiment 43

A computerized controller, comprising: a processor; a data input interface; a display; and a computer-readable memory, the computer readable memory holding instructions that, when executed by the processor, cause the computerized controller to read outputs produced by a set of sensors, wherein the sensors detect a magnetic field and the sensor outputs are usable to characterize the spatial relationship of a dental drill to the sensors; process the outputs to produce an indication of the spatial relationship of the dental drill to a patient's dentition; and produce a display of the indication of the spatial relationship of the dental drill to the patient's dentition.

### Embodiment 44

The computerized controller of embodiment 43, wherein the instructions, when executed by the processor, further cause the computerized controller to repeatedly update the display of the indication of the spatial relationship of the dental drill to the patient's dentition, substantially in real time.

### Embodiment 45

The computerized controller of any one of the embodiments 43-44, wherein the instructions, when executed by the processor, further cause the computerized controller to indicate on the display the location of the dental drill in relation to an implant shaft.

### Embodiment 46

The computerized controller of any one of the embodiments 43-45, wherein the instructions, when executed by the processor, further cause the computerized controller to: compare the location of the dental drill with the implant shaft; and produce a warning signal when the dental drill departs from the implant shaft by at least a predetermined amount.

### Embodiment 47

The computerized controller of embodiment 46, wherein the warning signal comprises one or more signals selected from the group consisting of a visual cue and a sound cue, alone or in any combination.

### Embodiment 48

A calibration station, comprising: a body defining a receptacle, wherein the receptacle is of a shape and size to receive a dental drill; and a plurality of sensors surrounding the receptacle, each sensor capable of producing an output when the sensor is exposed to a magnetic field associated with a dental drill placed in the receptacle.

### Embodiment 49

The calibration station of embodiment 48, wherein the sensors are positioned such that their outputs are capable of characterizing the shape and strength of the magnetic field.

### Embodiment 50

A non-transitory computer readable medium holding computer instructions adapted to be executed to implement a method of indicating the location of a dental drill, the method comprising: reading outputs produced by a set of sensors, wherein the sensors detect a magnetic field, and wherein the sensor outputs are usable to detect the location of a dental drill in relation to the sensors; processing the sensor outputs to produce an indication of the spatial relationship of the dental drill to a patient's dentition; and displaying the indication of the spatial relationship of the dental drill to the patient's dentition.

### Embodiment 51

A sensing device, comprising: a carrier having circuit traces, the carrier defining a through hole; and a plurality of electronic sensors mounted to the carrier around the through hole, each sensor being sensitive to a magnetic field and configured to produce an output indicating an aspect of the magnetic field; wherein the sensing device is of a size and shape for the sensors to fit within the mouth of a dental patient.

### Embodiment 52

The sensing device of embodiment 51, further comprising flexible electrical conductors configured to carry the sensor outputs outside the patient's mouth.

### Embodiment 53

The sensing device of any one of the embodiments 51-52, further comprising a wireless transmitter configured to transmit the sensor outputs outside the patient's mouth.

### Embodiment 54

The sensing device of embodiment 53, further comprising a battery that powers the sensors and the wireless transmitter.

### Embodiment 55

The sensing device of any one of the embodiments 51-54, wherein the plurality of sensors comprises at least six sensors, at least three of the sensors mounted to a first surface of the carrier, and at least three of the sensors mounted to a second surface of the carrier.

### Embodiment 56

The sensing device of any one of the embodiments 51-55, wherein the plurality of sensors comprises eight sensors, four of the sensors mounted to a first surface of the carrier, and four of the sensors mounted to a second surface of the carrier.

### Embodiment 57

A kit, comprising: a sensing device including: a carrier having circuit traces, the carrier defining a through hole; and a set of electronic sensors mounted to the carrier around the through hole, each sensor being sensitive to a magnetic field and configured to produce an output indicating an aspect of the magnetic field; wherein the sensing device is of a size and shape for the sensors to fit within the mouth of a dental patient; and a non-transitory computer readable medium holding computer instructions adapted to be executed to implement a method of indicating the location of a dental drill, the method including: reading outputs produced by the set of sensors, wherein the sensors detect a magnetic field, and wherein the sensor outputs are usable to detect the location of a dental drill in relation to the sensors; processing the sensor outputs to produce an indication of the spatial relationship of the dental drill to a patient's dentition; and displaying the indication of the spatial relationship of the dental drill to the patient's dentition.

### Embodiment 58

The kit of embodiment 57, further comprising a calibration station including: a body defining a receptacle, wherein the receptacle is of a shape and size to receive a dental drill; and a second set of sensors surrounding the receptacle, each sensor in the second set capable of producing an output when the sensor is exposed to a magnetic field associated with a dental drill placed in the receptacle.

### Embodiment 59

The kit of any one of the embodiments 57-58, further comprising an intermediate device configured to receive the sensor outputs and to relay the sensor outputs to a controller.

1-59. (canceled)

60. A system for indicating the location of a dental drill, the system comprising:
a dental handpiece comprising the dental drill; and
a plurality of sensors that detect a magnetic field and produce a set of respective sensor outputs, the sensor outputs usable at least in part to indicate the depth of the dental drill in relation to the plurality of sensors.

61. The system of claim 60, wherein the sensor outputs are further usable at least in part to indicate the lateral translational position of the drill in relation to the plurality of sensors, and the angular orientation of the drill in relation to the plurality of sensors.

62. The system of claim 60, wherein the dental drill is magnetized and generates the magnetic field.

63. The system of claim 60, further comprising a magnetic element that is fixed in relation to the dentition of a patient, and wherein the plurality of sensors is fixed in relation to the dental handpiece.

64. The system of claim 60, further comprising a workpiece guide registered to a patient's dentition, wherein the plurality of sensors is fixed in relation to the workpiece guide.

65. The system of claim 64, wherein the plurality of sensors is movable from a first fixed position in relation to the workpiece guide to a second fixed position in relation to the workpiece guide.

66. The system of claim 60, further comprising a carrier on which the plurality of sensors is mounted, at least three of the plurality of sensors mounted to a first surface of the carrier.

67. The system of claim 66, wherein the plurality of sensors comprises eight sensors, four of the plurality of sensors mounted to a first surface of the carrier and four of the plurality of sensors mounted to a second surface of the carrier, opposite the first surface.

68. The system of claim 60, further comprising a controller that receives the sensor outputs and processes the outputs to produce a visual indication of the spatial relationship of the dental drill to a patient's dentition, and repeatedly updates the indication of the spatial relationship of the dental drill to the patient's dentition, substantially in real time.

69. The system of claim 68, further comprising an intermediate device that receives the sensor outputs and relays the sensor outputs to the controller.

70. The system of claim 68, wherein the indication of the spatial relationship of the dental drill to the patient's dentition comprises:
a pictorial representation of the patient's dentition; and
a representation of the location of the dental drill location superimposed on the pictorial representation of the patient's dentition.

71. The system of claim 68, wherein the controller further produces an indication of the spatial relationship of the dental drill to a previously-specified implant shaft within the patient's dentition.

72. The system of claim 60, further comprising a calibration station that further includes:
a receptacle for the dental drill; and
a second plurality of sensors fixed in relation to the receptacle, each of the second plurality of sensors producing an output, and wherein the outputs of the second plurality of sensors are usable to characterize the spatial relationship of the magnetic field to the dental drill when the dental drill is placed in the receptacle.

73. A method of indicating the location of a dental drill, the method comprising:
reading outputs produced by a set of sensors, wherein the sensors detect a magnetic field, and wherein the sensor outputs are usable to detect the depth of a dental drill in relation to the sensors;
processing the sensor outputs to determine the depth of the dental drill in relation to the sensors;
producing, based on the sensor outputs, a visual indication of the spatial relationship of the dental drill to a patient's dentition;
displaying the visual indication of the spatial relationship of the dental drill to the patient's dentition; and
repeatedly updating the display of the visual indication of the spatial relationship of the dental drill to the patient's dentition, substantially in real time.

74. The method of claim 73, further comprising processing the sensor outputs to determine the translational position and the angular orientation of the dental drill in relation to the sensors.

75. The method of claim 73, further comprising, visually indicating on the display the location of the dental drill in relation to a previously-specified implant shaft.

76. A method, comprising:
fabricating a workpiece guide of a configuration to engage a dental arch of a particular patient having an implant site;
engaging the workpiece guide with the dental arch of the particular patient;
fixing a plurality of sensors to the workpiece guide, the plurality of sensors capable of, when the sensors are exposed to a magnetic field, producing a set of sensor outputs each indicating at least one aspect of the magnetic field;
bringing a dental handpiece comprising a dental drill into proximity with the plurality of sensors, wherein an element fixed to the handpiece produces a magnetic field, such that the plurality of sensors detects the magnetic field and produces the sensor outputs;
processing the sensor outputs to determine the depth of the dental drill in relation to the patient's dentition; and
displaying, on a visual display, an indication of the depth of the dental drill to the patient's dentition.

77. The method of 76, further comprising simultaneously displaying, on the visual display, a visual indication of the spatial relationship of the dental drill to a desired implant shaft.

78. A sensing device, comprising:
a carrier having circuit traces, the carrier defining a through hole; and
a plurality of electronic sensors mounted to the carrier around the through hole, each sensor being sensitive to a magnetic field and configured to produce an output indicating an aspect of the magnetic field;
wherein the sensing device is of a size and shape for the sensors to fit within the mouth of a dental patient.

79. The sensing device of claim 78, further comprising flexible electrical conductors configured to carry the sensor outputs outside the patient's mouth.

80. The sensing device of claim 78, further comprising a wireless transmitter configured to transmit the sensor outputs outside the patient's mouth.

81. The sensing device of claim 80, further comprising a battery that powers the sensors and the wireless transmitter

82. The sensing device of claim 78, wherein the plurality of sensors comprises at least three of the sensors mounted to a first surface of the carrier.

83. The sensing device of claim 78, wherein the plurality of sensors comprises eight sensors, four of the sensors mounted to a first surface of the carrier, and four of the sensors mounted to a second surface of the carrier.
